(19) United States
(12) Patent Application Publication
(10) Pub. No.: US 2025/0075267 A1
(43) Pub. Date: Mar. 6, 2025

(54) METHODS AND COMPOSITIONS FOR LIGATION AND SAMPLE ANALYSIS

(71) Applicant: 10x Genomics, Inc., Pleasanton, CA (US)

(72) Inventors: Ashley HAYES, Oakland, CA (US); Robert HENLEY, Castro Valley, CA (US)

(21) Appl. No.: 18/821,061

(22) Filed: Aug. 30, 2024

Related U.S. Application Data
(60) Provisional application No. 63/580,181, filed on Sep. 1, 2023.

(51) Int. Cl. C12Q 1/6855; C12Q 1/48; C12Q 1/682; C12Q 1/6841; C12Q 1/6869

(52) U.S. Cl. CPC C12Q 1/6855; C12Q 1/485; C12Q 1/682; C12Q 1/6841; C12Q 1/6869; G01N 2333/91215

(57) ABSTRACT

The present disclosure relates in some aspects to methods for analyzing a target nucleic acid in a biological sample (e.g., cell or tissue sample). In some aspects, provided herein are methods and compositions for analyzing a target nucleic acid in a ligation-based assay using one or more probes, and phosphorylation of one or more probes hybridized to a target nucleic acid is performed.

### CROSS-REFERENCE TO RELATED APPLICATIONS

This application claims priority to U.S. Provisional Patent Application No. 63/580,181 filed on Sep. 1, 2023 entitled “METHODS AND COMPOSITIONS FOR LIGATION AND SAMPLE ANALYSIS,” which is herein incorporated by reference in its entirety for all purposes.

### FIELD

The present disclosure relates in some aspects to methods and compositions for processing nucleic acid molecules during analysis of a sample, such as detection of a nucleic acid sequence in situ in a biological sample, e.g., in a cell or tissue sample, or on a substrate comprising spatially barcoded oligonucleotides.

### BACKGROUND

Methods are available for analyzing nucleic acids present in a biological sample, such as a cell or a tissue. However, probe-based multiplexed analysis of a panel of transcripts has remained challenging and assays may require costly reagents. Improved methods for analyzing nucleic acids present in a biological sample are needed. Provided herein are methods and compositions that address such and other needs.

### BRIEF SUMMARY

Provided in some embodiments herein are methods for analyzing a target nucleic acid, comprising contacting the target nucleic acid in a cell or tissue sample with a probe or probe set lacking phosphorylated 5′ ends, wherein the target nucleic acid comprises a target region and the probe or probe set comprises a hybridization region, and the hybridization region hybridizes to the target region in the cell or tissue sample; contacting the cell or tissue sample with a kinase to phosphorylate one or more ends of the probe or probe set hybridized to the target nucleic acid; ligating the probe or probe set to form a circularized probe; amplifying the circularized probe; and detecting an amplification product of the circularized probe generated in the cell or tissue sample. In some embodiments, ligation of the probe or probe set comprises a RNA templated ligation. In some embodiments, ligation of the probe or probe set comprises a DNA templated ligation.

In some embodiments, a wash is performed to remove unbound probes or probe sets prior to contacting the cell or tissue sample with the kinase. In some embodiments, the wash is performed using a ligation buffer comprising a salt and ATP. In some cases, the ligation buffer is at about pH 8.0.

In some embodiments, the ligation of the probe or probe set uses the target nucleic acid as a template, with or without gap filling prior to the ligation. In some embodiments, the phosphorylation of the one or more ends of the probe or probe set is performed in a buffer comprising ATP. In some cases, the phosphorylation of the one or more ends of the probe or probe set is performed in a ligation buffer. In some examples, the cell or tissue sample is incubated with the kinase (e.g., a polynucleotide kinase) at about 30° C. to about 40° C. In some examples, the cell or tissue sample is incubated with the kinase (e.g., a polynucleotide kinase) at about 37° C. In some embodiments, the biological sample is incubated with the kinase for at least 15 minutes, at least 30 minutes, at least 1 hour or at least 2 hours. In some embodiments, the kinase is a T7 or T4 Polynucleotide Kinase (PNK).

In some embodiments, the probe is a linear probe comprising a split hybridization region comprising a first hybridization region at the 5′ end of the probe and a second hybridization region at the 3′ end of the probe. In some embodiments, a probe set is used comprising a first probe and a second probe, wherein hybridization region of the probe set comprises a split hybridization region comprising a first hybridization region comprised by the first probe of the probe set and a second hybridization comprised by the second probe of the probe set.

In some embodiments, the ligation comprises enzymatic ligation, chemical ligation, template dependent ligation, and/or template independent ligation. In some embodiments, the cell or tissue sample is contacted with a splint nucleic acid prior to ligating the probe or probe set. In some cases, the cell or tissue sample is contacted with the splint nucleic acid after contacting the target nucleic with the probe or probe set. In some embodiments, ligation of the probe set comprises two or more ligations. In some examples, the two or more ligations comprise a RNA templated ligation and a DNA templated ligation. In some cases, the two or more ligations are performed using different ligases. In some embodiments, the two or more ligations are performed separately and sequentially. In some embodiments, the two or more ligations comprise: a first ligation wherein a 5′ end of the first probe is ligated to a 3′ end of the second probe using the target nucleic acid as a first template, and a second ligation wherein a 3′ end of the first probe is ligated to a 5′ end of the second probe using the splint nucleic acid as a second template. In some cases, the first ligation is RNA templated ligation, the second ligation is DNA templated ligation, and the first ligation is performed prior to the second ligation.

In some embodiments, the probe or probe set comprises one or more barcode sequences. In some embodiments, the amplification product comprises the one or more barcode sequences or complementary sequences thereof. In some examples, the amplification product of the circularized probe is a rolling circle amplification (RCA) product. In some embodiments, detecting the amplification product of the circularized probe comprises detecting the one or more barcode sequences or complementary sequences thereof. In some embodiments, the amplification product comprises multiple copies of each of the one or more barcode sequences or complements thereof.

In some embodiments, the cell or tissue sample comprises a plurality of target nucleic acids and the cell or tissue sample is contacted with a plurality of probe or probe sets, wherein each probe or probe set of the plurality of probes or probe sets corresponds to a target nucleic acid of the plurality of target nucleic acids.

In some embodiments, detecting the one or more barcode sequences or complements thereof comprises: contacting the cell or tissue sample with a universal pool of detectably labeled probes and a first pool of intermediate probes, wherein intermediate probes of the first pool of intermediate probes comprise hybridization regions complementary to the one or more barcode sequences or complements thereof, and reporter regions complementary to a detectably labeled probe of the universal pool of detectably labeled probes; detecting complexes formed between the one or more barcode sequences or complements thereof, the intermediate probes of the first pool of intermediate probes, and the detectably labeled probes; and removing the intermediate probes of the first pool of intermediate probes and the detectably labeled probes. In some cases, each of the one or more barcode sequences or complement thereof is assigned a corresponding series of signal codes that identifies the barcode sequence or complement thereof, and wherein detecting the one or more barcode sequences or complements thereof comprises decoding the barcode sequences or complements thereof by detecting the corresponding series of signal codes using sequential hybridization, detection, and removal of sequential pools of intermediate probes and the universal pool of detectably labeled probes. In some aspects, each series of signal codes is a fluorophore sequence assigned to the corresponding barcode sequence or complement thereof of the one or more barcode sequences of complements thereof. In some embodiments, the detectably labeled probes are fluorescently labeled.

In some embodiments, the target nucleic acid is at a location in the cell or tissue sample and the amplification product of the circularized probe is generated and detected at the location in the cell or tissue sample.

Provided herein is a method for analyzing a target nucleic acid in a cell or tissue sample, comprising a) contacting the cell or tissue sample with a plurality of probe sets, wherein each probe set of the plurality of probe sets comprises a first probe and a second probe, wherein the target nucleic acid comprises a first target region and a second target region and the first probe and second hybridizes to the first and second target regions, respectively; b) contacting the cell or tissue sample with a kinase to phosphorylate the 5′ ends of the first probes and/or the second probes hybridized to the target nucleic acid; c) ligating the first probes to the second probes hybridized to the target nucleic acid to form a plurality of ligated probes in the cell or tissue sample; and d) detecting the plurality of ligated probes or a product thereof.

In some embodiments, the ligating comprises a first ligation using the target nucleic acid as a template. In some embodiments, the first ligation is an RNA templated ligation. In some embodiments, first and second probes that are not hybridized to the target nucleic acid are removed from the cell or tissue sample. In some embodiments, the removing comprises one or more washes prior to contacting the biological sample with the kinase. In some embodiments, the one or more washes is performed using a wash buffer comprising a salt and ATP. In some embodiments, the wash buffer is at about pH 8.0.

In some embodiments, each of the plurality of ligated probes is a circularized probe. In some embodiments, the ligated probes of the plurality of ligated probes are linear. In some embodiments, the ligating further comprises a second ligation using a splint nucleic acid as a template. In some examples, the cell or tissue sample is contacted with the splint nucleic acid prior to ligating the first probes and the second probes. In some cases, the cell or tissue sample is contacted with the splint nucleic acid after contacting the target nucleic acid with the plurality of probe sets. In some embodiments, the second ligation is a DNA templated ligation.

In some embodiments, the method comprises amplifying the circularized probes. In some embodiments, the method further comprises detecting an amplification product of each of the circularized probes. In some embodiments, the amplifying comprises rolling circle amplification (RCA). In some cases, the method comprises gap filling prior to the ligating the first probes to the second probes.

In some embodiments, phosphorylation of the 5′ ends of the first probes and/or the 5′ ends of the second probes is performed in a buffer comprising ATP. In some cases, the phosphorylation of the 5′ ends of the first probes and/or the 5′ ends of the second probes is performed in a ligation buffer. In some embodiments, the method further comprises gap filling prior to the ligating the 5′ ends of the first probes and/or the 5′ ends of the second probes. In some embodiments, the cell or tissue sample is incubated with the kinase at about 30° C. to about 40° C. In some embodiments, the cell or tissue sample is incubated with the kinase at about 37° C. In some embodiments, the cell or tissue sample is incubated with the kinase for at least 15 minutes, at least 30 minutes, at least 1 hour or at least 2 hours.

In some embodiments, the kinase is a T7 or T4 Polynucleotide Kinase (PNK).

In some embodiments, the plurality of ligated probes or a product thereof is detected on an array and/or by nucleic acid sequencing. In some embodiments, the target nucleic acid is at a location in a biological sample and a ligated probe of the plurality of ligated probes is generated at the location in the biological sample, and wherein the ligated probe of the plurality of ligated probes and/or a product thereof is detected at the location in the cell or tissue sample. In some embodiments, the target nucleic acid is at a location in the cell or tissue sample and a ligated probe of ligated probes is generated at the location in the biological sample, and wherein a ligated probe of the plurality of ligated probes and/or the product thereof is covalently or noncovalently attached to an oligonucleotide immobilized on a substrate. In some embodiments, the oligonucleotide comprises a spatial barcode sequence. In some embodiments, the oligonucleotide comprises a capture sequence complementary to a sequence of the ligated probe and/or the product thereof.

In some embodiments, the method comprises generating a spatially barcoded oligonucleotide comprising (i) a sequence of the ligated probe or product thereof or complement thereof and (ii) a sequence of the spatial barcode sequence or complement thereof. In some embodiments, the spatially barcoded oligonucleotide is released from the substrate. In some aspects, the released spatially barcoded oligonucleotide is sequenced, thereby detecting the target nucleic acid or sequence thereof at the spatial location in the biological sample.

In some embodiments, the target nucleic acid is RNA. In some embodiments, the target nucleic acid is endogenous or generated in situ in the sample. In some embodiments, the target nucleic acid is a product or derivative of an endogenous molecule in the cell or tissue sample. In some embodiments, the target nucleic acid is comprised in a labelling agent that directly or indirectly binds to an analyte in the sample, or is comprised in a product of the labelling agent. In some embodiments, the labelling agent comprises a protein, a peptide, an antibody or an epitope binding fragment thereof, a lipophilic moiety, a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. In some embodiments, the lipophilic moiety is cholesterol. In some embodiments, the labelling agent comprises a reporter oligonucleotide.

In some embodiments, the target nucleic acid is immobilized in the cell or tissue sample. In some cases, the target nucleic acid is crosslinked to one or more molecules in the cell or tissue sample, a matrix such as a hydrogel, and/or one or more functional groups on a substrate. In some embodiments, the detecting comprises imaging the cell or tissue sample to detect signals associated with the target nucleic acid in situ.

In some embodiments, the cell or tissue sample is a processed or cleared cell or tissue sample. In some embodiments, the cell or tissue sample is embedded in a hydrogel.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

The following drawings illustrate certain embodiments of the features and advantages of this disclosure. These embodiments are not intended to limit the scope of the appended claims in any manner.

FIG. 1 depicts an example workflow for amplification of a probe set hybridized to a target nucleic acid comprising in situ phosphorylation and ligation of the probe set.

FIG. 2A shows exemplary images of signals detected from circularizable probes (e.g. as depicted in diagram of FIG. 2B, top) hybridized to a target nucleic acid that were synthesized with a 5′ phosphate (“5′ Phos Mod”; left), synthesized with a 5′ phosphate and treated with Calf-intestinal alkaline phosphatase (CIP) (“5′ Phos Mod+CIP”; middle), or treated with CIP and subsequently phosphorylated using T4 polynucleotide kinase (T4 PNK) in situ (“CIP+T4 PNK”; right), and subsequently subjected to conditions for ligation, amplification, and detection. FIG. 2B shows images of detected probes treated with T4 PNK in the sample after washing with PBST or ligation buffer.

FIG. 3 shows results from detection of analytes using circularizable probes or probe sets for indicated conditions, including following in situ phosphorylation of the circularizable probes or probe sets.

FIG. 4 shows exemplary imaging results from circularizable 5′ phosphorylated probes (positive control; left), and results from CIP-treated probes lacking 5′ phosphates that were subsequently hybridized and phosphorylated in situ using the indicated concentrations of T4 PNK. Probes were subsequently subjected to conditions for ligation, amplification, and detection.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Methods for detecting a target nucleic acid using the target nucleic acid as a template for the ligation of a probe or probe sets are available. For example, various probe or probe sets may be used to hybridize to and detect a target nucleic acid. In some cases, circularizable probes can be linear nucleic acid molecules that comprise sequences complementary to the target nucleic acid at their 5′ and 3′ ends. Upon hybridization of a circularizable probe to its target nucleic acid, the 5′ and 3′ ends are brought in proximity to enable ligation. In some cases, a probe set may comprise two or more linear nucleic acid molecules that collectively comprise sequences complementary to the target nucleic acid and upon hybridization of the linear nucleic acid molecules of the probe set to the target nucleic acid, the hybridized regions are brought in proximity to enable ligation to form a ligated probe molecule. Ligation-based assays can be used to detect a plurality of target nucleic acids in a multiplexed assay. A panel of probes (e.g., circularizable probes or probe sets) can be designed to hybridize to target sequences in the target nucleic acids and ligation can be performed to allow specific detection of the analytes. However, probe-based multiplexed analysis of a panel of transcripts may require costly reagents, such as a large number of probes with ligatable ends. For example, the cost difference between nucleic acid probes synthesized without modifications and nucleic acid probes synthesized with a 5′ phosphate modification to allow for ligation may be about 30% to 50% of the cost.

The present disclosure provides methods and compositions for analysis of target nucleic acids in a sample by hybridizing a probe or probe set comprising a non-phosphorylated 5′ end to a target nucleic acid, contacting the sample with a kinase to phosphorylate one or more ends of the probe or probe set hybridized to the target nucleic acid, and ligating the probe or probe set in the biological sample. In some aspects, compared to using a large number of different probes synthesized with a 5′ phosphate modification prior to an in situ detection assay, using the same probes synthesized without the 5′ phosphate modification and phosphorylating the probes in a single reaction in situ, as part of the in situ detection assay workflow, can drastically reduce the cost of the assay. In some aspects, compared to using probes synthesized with a 5′ phosphate modification, the phosphorylation of only probes or probe sets that hybridized to the target nucleic acid can reduce the cost of the assay. Rather than ensuring that all probes contacted with the sample have a phosphate modification, the provided methods can allow only a subset of probes contacted with the sample that is hybridized to the target nucleic acid to be treated with the kinase. In some cases, unbound probes or probe sets can be removed from the sample prior to contacting the sample with the kinase. In some aspects, the methods described herein involving in situ phosphorylation of hybridized probes can provide advantages including high specificity and/or increased specificity in comparison to different assays. In some embodiments, the analysis is an in situ analysis. In some embodiments, the detection of a sequence described herein can provide information regarding the location and/or identity of an associated target nucleic acid (e.g., hybridized by the probe(s)) in a sample. In some embodiments, target nucleic acid(s) are in a cell or tissue sample where diffusion of reagents such as probes and enzymes affect efficiency of the reactions in the sample. The methods of the present disclosure are not limited to in situ detection of analytes and analysis. In some embodiments, the phosphorylation of probes or probe sets described herein are performed on probes in a tissue sample or a section of a cell pellet or cell block. In some embodiments, the phosphorylation of probes or probe sets described herein are not performed in vitro or in a fluid comprising isolated analytes. In some embodiments, the phosphorylation of probes or probe sets is performed on cells or a tissue sample on a substrate.

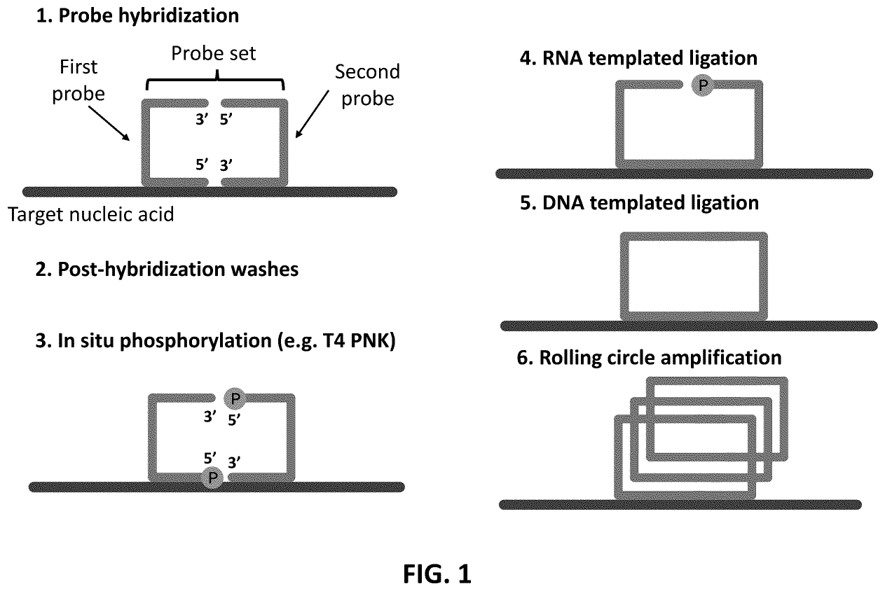

In some embodiments, a probe (e.g., a circularizable probe, or a first and second probe in a probe set) is treated in the biological sample by a kinase to add a 5′ phosphate to the the probe. In some aspects, the kinase catalyzes the transfer of the gamma-phosphate from ATP to the 5′—OH group of the nucleic acid probe. As shown in FIG. 1, a first and second probe in a probe set (e.g., a circularizable probe set) can be treated with a kinase to add a phosphate to each 5′ end of the first and second probes. Similarly, a probe (e.g., a circularizable probe) can be treated with a kinase to add a phosphate to the 5′ end of the probe. In some aspects, a probe set comprising a first linear probe and a second linear probe can be ligated at one end of each molecule to generate a linear ligated-probe molecule.

In some embodiments, the method comprises hybridizing a circularizable probe or probe set disclosed herein to a target mRNA molecule in the biological sample. In some embodiments, the method comprises hybridizing a probe or probe set disclosed herein to a target mRNA molecule in the biological sample. In some embodiments, the method comprises hybridizing a linear probe set, such as any disclosed herein, to a target mRNA molecule in the biological sample. In some embodiments, the two hybridized linear probes are ligated together after the biological sample is treated with the kinase. In some embodiments, after kinase treatment of the hybridized probe(s), an RNA templated DNA ligation is performed. In some embodiments, after kinase treatment of the hybridized probe(s), a DNA templated ligation is performed. In some embodiments, after kinase treatment of the hybridized probe(s), an RNA templated ligation and a DNA templated ligation is performed. In some embodiments, the RNA templated ligation comprises ligation of the probe or probe set using the target nucleic acid, which is an RNA molecule, as template. In some embodiments, the DNA templated ligation comprises ligation of the probe or probe set using a splint nucleic acid, which is a DNA molecule, as template.

Provided herein are circularizable probes for in situ applications and/or spatial analysis applications. In some embodiments, only probes that are hybridized and treated with the kinase have a 5′ phosphate that are used for downstream ligation and are detected. In some embodiments, a plurality of probes hybridized to different target nucleic acids are treated with the kinase and are used for downstream ligation and detection.

The present disclosure also provides probes, sets of probes, compositions, kits, systems, and devices for use in accordance with the provided methods. In some aspects, the provided methods and systems can be applied to detect, image, quantitate, or determine the presence or absence of one or more target nucleic acid(s) or portions thereof. In some aspects, the provided methods can be applied to detect, image, quantitate, or determine the sequence of one or more target nucleic acid(s).

In some aspects, the provided embodiments can be employed for in situ detection and/or sequencing of a target nucleic acid in a cell, e.g., in cells of a biological sample or a sample derived from a biological sample, such as a tissue section on a solid support, such as on a transparent slide. In some aspects, the provided embodiments can be employed for sequencing of a target nucleic acid in a deposited cell sample on a solid support, such as on a transparent slide.

In some aspects, provided herein are in situ assays using microscopy as a readout, e.g., nucleic acid sequencing, hybridization, or other detection or determination methods involving an optical readout. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof generated using the provided probes. In some embodiments, the amplification product or portion thereof is an RCA product. In some embodiments, the provided probes are circularizable probes or probe sets. In some embodiments, the provided probes are linear probe sets. In some embodiments, a method for spatially profiling analytes such as the transcriptome or a subset or portion thereof in a biological sample is provided. Methods, compositions, kits, devices, and systems for these in situ assays, spatial genomics and transcriptomics assays, are provided. In some embodiments, a provided method is quantitative and preserves the spatial information within a tissue sample without physically isolating cells or using homogenates.

In some embodiments, through the use of various probes and processing workflows (e.g., various circularizable probes and/or linear probe such as described in Section II), the present disclosure provides methods for high-throughput profiling a large number of targets in situ, such as transcripts and/or DNA loci, for detecting and/or quantifying nucleic acids in cells, tissues, organs or organisms. In some embodiments, the various probes are various circularizable probes such as described in Section II. In some embodiments, the various probes are various linear probes such as described in Section II.

In some aspects, the methods disclosed herein involve the use of one or more probes or probe sets that hybridize to a target nucleic acid, such as an RNA molecule, wherein after hybridization of the probe or probe set to the target nucleic acid, the probes are treated with a kinase. The specific probe or probe set design can vary. In some embodiments, the methods disclosed herein are ligation dependent and involve only those probes that have been treated by a kinase and thus have a 5′ phosphate available for a ligation reaction. In some embodiments, the ligatable probes are ligated. For example, either the two ends of a single circularizable probe can be ligated or two separate probes of a probe set can be ligated together. In some embodiments, a primary probe ligated to form a circular template (e.g., a DNA probe that directly binds to an RNA target) is amplified through rolling circle amplification (RCA).

In some embodiments, the provided methods involve analyzing, e.g., detecting or determining, one or more sequences present in a probe or probe set and/or in an amplification or extension product of a ligated probe or probe set, such as in an amplification product of a circularized probe. In some embodiments, the analysis is used to correlate a sequence detected in an amplification product to a probe or probe set or a first and/or second probe used to form a ligated probe. In some embodiments, the detection of a sequence in an amplification product provides information regarding the location and/or identity of an associated target nucleic acid (e.g., hybridized by the circularizable probe or first and second probe) in a sample. In some embodiments, due to amplification of one or more polynucleotides (e.g., a circularized probe or probe set), particular sequences present in the amplification product are detected even when the template (e.g., the circularized probe or probe set) is present at low levels before the amplification. In some embodiments, the one or more polynucleotides is a circularized probe or probe set and the template is the template nucleic acid.

In some aspects, the provided methods can be applied for various applications, such as for in situ analysis, comprising in situ detection (e.g., based on hybridization such as sequential hybridization) and/or sequencing of target nucleic acids and multiplexed nucleic acid analysis. In some aspects, the provided methods can be used with a spatial array.

In some embodiments, provided herein are methods for assessing one or more target nucleic acids, such as a plurality of different mRNAs, in a biological sample, such as a cell or a tissue sample (such as a tissue section). In some embodiments, the target nucleic acid comprises RNA. In some embodiments, the target nucleic acid is an mRNA. In some embodiments, the probe or probe set comprises DNA.

In some aspects, the provided methods are employed for in situ analysis of target nucleic acids, for example for in situ sequencing or multiplexed analysis in intact tissues or a sample with preserved cellular or tissue structure.

In some aspects, the provided methods comprise one or more steps of ligating the polynucleotides, for instance of ligating the ends of a circularizable probe to form a circularized probe and/or ligating a first probe and second probe to form a ligated linear probe product. In some aspects, the provided methods involve a step of amplifying one or more of the polynucleotides (e.g., a circularized probe produced therefrom), to generate an amplification product. In some aspects, the provided methods involve a step of detecting and/or determining the sequence of all or a portion of the amplification product. In some aspects, the provided methods involve performing one or more of the steps described herein, simultaneously and/or sequentially.

Particular aspects and steps of the methods can be carried out as described herein, for example in Sections II-V; and/or using any suitable processes and methods for carrying out the particular aspects and steps.

### II. Probes and Ligation

Disclosed herein in some aspects are nucleic acid probes and/or probe sets that are introduced into a cell or used to otherwise contact a biological sample such as a tissue sample or a sample comprising cells. In some aspects, the nucleic acid probes and/or probe sets comprise a hybridization region capable of hybridizing to target sequences in the target nucleic acid. In some aspects, the target nucleic acid comprises RNA and the probe or probe set comprises DNA and/or RNA. In some aspects, the target nucleic acid comprises RNA and the probe or probe set comprises DNA. In some aspects, the probe or probe set comprises a split hybridization region comprising a first hybridization region at the 5′ end of the probe and the second hybridization region at the 3′ end of the probe. In some embodiments, the probe set comprises a hybridization region that is a split hybridization region comprising a first hybridization region comprised by a first probe of the probe set and second hybridization region comprised by a second probe of the probe set, and the target nucleic acid is contacted with the probe set comprising the first and second probes. In some embodiments, the probe or probe set comprises a non-phosphorylated 5′ end. In some cases, the one or more probes or probe sets lacks phosphorylated 5′ ends. In some embodiments, the probe set comprises a first probe and a second probe that each comprises a non-phosphorylated 5′ end. Compared to an assay that uses probes or probe sets synthesized with 5′ phosphate modifications, the kinase treatment after hybridization in the methods provided herein can reduce costs. In addition, the probes or probe sets can be less challenging to manufacture.

The probes may comprise any one of a variety of entities that can hybridize to a nucleic acid, typically by Watson-Crick base pairing, such as DNA, RNA, LNA, PNA, etc., depending on the application. The nucleic acid probe(s) typically contains a hybridization region that is able to bind to at least a portion of a target nucleic acid, in some embodiments specifically. The nucleic acid probe may be able to bind to a specific target nucleic acid (e.g., an mRNA, or other nucleic acids as discussed herein). In some embodiments, the nucleic acid probes are detected using a detectable label, and/or by using secondary nucleic acid probes able to bind to the nucleic acid probes. In some embodiments, the nucleic acid probes are compatible with one or more biological and/or chemical reactions. For instance, a nucleic acid probe disclosed herein can serve as a template or primer for a polymerase, a template or substrate for a ligase, a substrate for a click chemistry reaction, and/or a substrate for phosphorylation.

Provided herein are methods involving the use of one or more probes (e.g., a circularizable probe such as a padlock probe) for analyzing one or more target nucleic acid(s), such as a target nucleic acid (for example, a messenger RNA) present in a cell or a biological sample, such as a tissue sample. Also provided are probes, sets of probes, compositions, kits, systems and devices for use in accordance with the provided methods. In some aspects, the provided methods and systems can be applied to detect, image, quantitate, or determine the presence or absence of one or more target nucleic acid(s) or portions thereof. In some aspects, the provided methods can be applied to detect, image, quantitate, or determine the sequence of one or more target nucleic acid(s).

In some aspects, a target nucleic acid disclosed herein comprises any polynucleotide nucleic acid molecule (e.g., DNA molecule; RNA molecule, modified nucleic acid, etc.) for assessment in accordance with the provided embodiments, such as a polynucleotide present in a cell. In some embodiments, the target nucleic acid is a coding RNA. In some embodiments, the coding RNA is mRNA. In some embodiments, the target nucleic acid is a single RNA molecule. In other embodiments, the target is at least one RNA molecule. In some embodiments, the at least one RNA molecule is a group of 2, 3, 4, 5, 6 or more RNA molecules. These RNA molecules may differ in molecule type, and/or may differ in sequence. In some embodiments, the target nucleic acid is a non-coding RNA. In some embodiments, the non-coding RNA is a tRNA, rRNA, microRNA (miRNA), mature miRNA or immature miRNA. In some embodiments, the target nucleic acid is a splice variant of an RNA molecule in the context of a cell. In some embodiments, the splice variant of an RNA molecule is an mRNA or a pre-mRNA. A suitable target nucleic acid can therefore be an unspliced RNA (e.g., pre-mRNA, mRNA), a partially spliced RNA, or a fully spliced RNA, etc. Target nucleic acids of interest may be variably expressed, e.g., have a differing abundance, within a cell population, wherein the methods of the present disclosure allow profiling and comparison of the expression levels of nucleic acids, comprising but not limited to, RNA transcripts, in individual cells. A target nucleic acid can also be a DNA molecule, e.g., a denatured genomic, viral, plasmid, etc. For example, the methods can be used to detect copy number variants, e.g., in a cancer cell population in which a target nucleic acid is present at different abundance in the genome of cells in the population; a virus-infected cells to determine the virus load and kinetics, and the like.

In some aspects, the methods provided herein are used to analyze a target nucleic acid. In some embodiments, the target nucleic acid is an endogenous nucleic acid present in a biological sample. In some embodiments, the target nucleic acid is present in a cell, in a tissue, in a cell pellet, in a cell block, or from a tissue sample. In some embodiments, the tissue sample is a tissue section. In some embodiments, the tissue sample is an intact tissue sample or a non-homogenized tissue sample. In some embodiments, the tissue sample is a fresh tissue sample. In some embodiments, the tissue has previously been processed (i.e. is a processed tissue). In some embodiments, the processed tissue is fixed, embedded, frozen, and/or permeabilized. Various tissue processing methods that may be used in connection with the methods described herein are described in Section V.

In some aspects, the provided embodiments can be employed for in situ detection and/or sequencing of a target nucleic acid in a cell, e.g., in cells of a biological sample or a sample derived from a biological sample, such as a tissue section on a solid support, such as on a transparent slide.

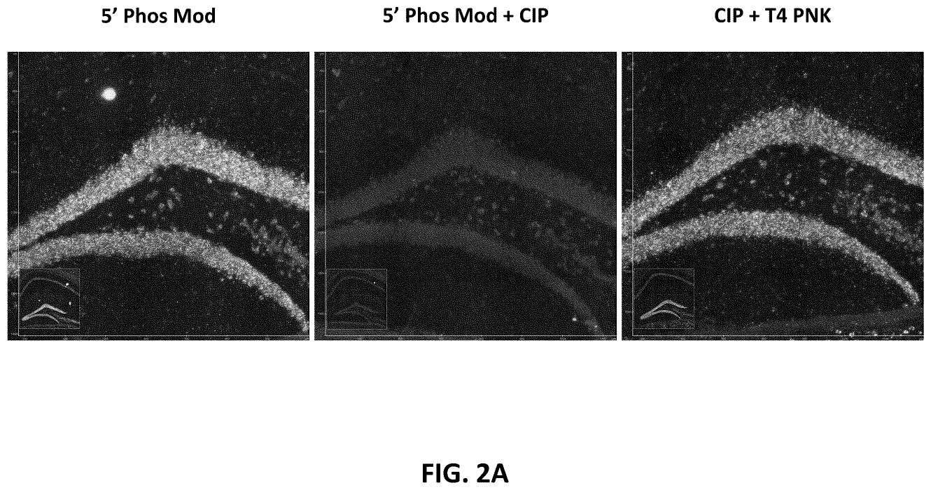

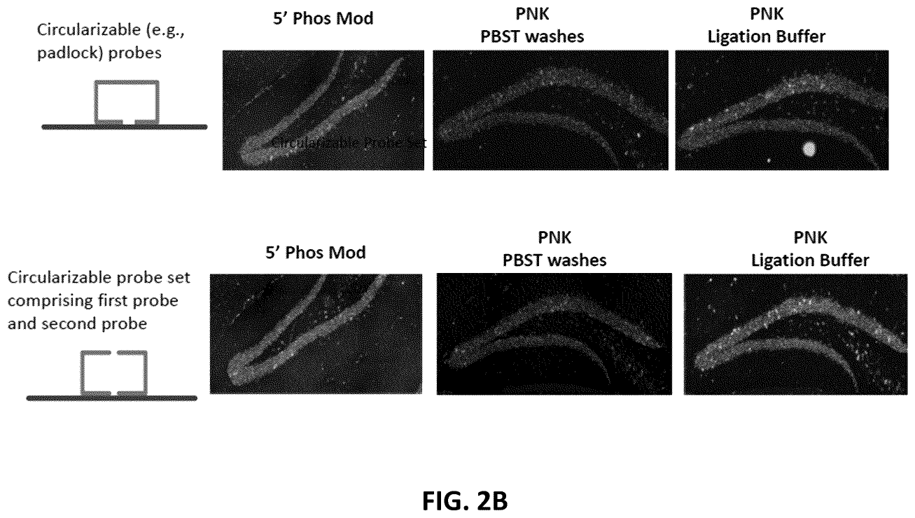

In some aspects, the methods disclosed herein involve the use of one or more probes or probe sets that hybridize to a target nucleic acid, such as an RNA molecule, wherein at least one probe is a circularizable probe comprising a hybridization region that hybridizes to a target nucleic acid. The specific probe or probe set design can vary (e.g., circularizable probe or a pair of linear probes). For example, as shown in FIG. 1, the probe set can comprise two probe molecules (e.g., a first probe and a second probe). The first probe and the second probe can each comprise a portion of the split hybridization region that hybridizes to the target region in the target nucleic acid. Alternatively, the probe can be a single molecule (e.g., a circularizable probe) with the split hybridization regions at the 5′ and 3′ ends of the circularizable probe (e.g. as shown in FIG. 2B; top). The probe(s) can be contacted with a target nucleic acid (for example, RNA). The probe or probe set can comprise two hybridization regions complementary to sequences on the target nucleic acid. Upon hybridization, the ends of the probe or probe set can be positioned between the two split hybridization regions of the probe(s). Upon treatment with a kinase, the 5′ end of the probe(s) is phosphorylated and the ends are then capable of being ligated together. In some embodiments, prior to ligation, the 5′ and 3′ ends can optionally be extended, such as by a polymerase, using the RNA target nucleic acid as a template. The ends of the probe(s) can be ligated using a ligase (such as a T4 DNA ligase, a PBCV-1 DNA ligase, or a T4 RNA ligase 2 (T4 Rn12)). In some embodiments, the ligase is T4 DNA ligase, a PBCV-1 DNA ligase, or a T4 RNA ligase 2 (T4 Rn12). The probe(s) can be ligated to circularize the circularizable probe or probe set. The ligated probe can then be amplified to generate a rolling circle amplification product (RCP) and then detected. In some instances, a probe set comprising the linear first and second probes can be ligated together to form a linear ligated probe (e.g., ligated product of the first and second probe).

In some embodiments, a circularized probe or probe set (e.g., a DNA probe that directly binds to an RNA target) is amplified through rolling circle amplification (RCA). In some embodiments a circularizable probe or probe set is ligated, circularized, and amplified. In some embodiments the circularizable probe or probe set is ligated using a target nucleic acid as a template. In some embodiments, the target nucleic acid is an RNA. In some embodiments, the probe or probe set contains one or more barcodes. In some embodiments, the one or more barcodes are indicative of a sequence in the target nucleic acid.

In some aspects, the provided methods involve analyzing e.g., detecting or determining, one or more sequences present in the polynucleotides and/or in an amplification product, such as in an amplification product of a circularized probe, which may comprise one or more barcode sequences. In some embodiments, the analysis comprises detecting or determining one or more sequences present in the polynucleotides and/or in the amplification product. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region (e.g., a single nucleotide) of interest in a target nucleic acid. In some embodiments, the region is a single nucleotide. In some embodiments, the analysis is used to correlate a sequence detected in an amplification product to a circularizable probe. In some embodiments, the correlation is via one or more barcodes. In some embodiments, the detection of a sequence in an amplification product provides information regarding the location and/or identity of an associated target nucleic acid (e.g., hybridized by the probe(s)) in a sample. In some embodiments, due to amplification of one or more polynucleotides (e.g., a circularizable probe), particular sequences present in the amplification product or complementary sequences thereof can be detected even when a polynucleotide is present at low levels before the amplification. For example, the number of copies of the barcode sequence(s) and/or a complementary sequence thereof is increased by virtue of the amplification of a probe comprising the barcode sequence(s) and/or complementary sequence thereof, thereby enabling specific and sensitive detection of a signal indicative of the identity of a short region (e.g., a single nucleotide) of interest in a target nucleic acid. In particular embodiments, the amplification product is an in situ rolling circle amplification (RCA) product of a circularized probe.

In some embodiments, provided herein are methods for assessing one or more target nucleic acids, such as a plurality of different mRNAs, in a biological sample, such as a cell or a tissue sample (such as a tissue section). In some embodiments, the target nucleic acid comprises DNA. In some embodiments, the target nucleic acid comprises RNA. In some embodiments, the target nucleic acid comprises mRNA. In some embodiments, the probe comprises DNA. In some embodiments, the target nucleic acid is RNA and the probe comprises DNA.

In some aspects, the provided methods involve a step of contacting, or hybridizing, one or more polynucleotides, such as any one of the probes described herein, to a cell or a sample containing a target nucleic acid with a target region. In some aspects, the provided methods comprise contacting the cell or tissue sample with a kinase to phosphorylate one or more ends of the probe or probe set hybridized to the target nucleic acid. In some aspects, the provided methods comprise one or more steps of ligating the ligatable or ligatable ends of polynucleotides, for instance of ligating the ligatable ends of a circularizable probe to form a circularized probe. In some aspects, the one or more steps of ligating the ligatable or ligatable ends of polynucleotides, comprise ligating the ligatable ends of a probe set to form a linear ligated probe. In some aspects, the provided methods involve a step of amplifying one of the polynucleotides (e.g., a padlock probe, a circularized probe or a linear probe produced therefrom), to generate an amplification product. In some aspects, the provided methods involve a step of detecting and/or determining the sequence of all or a portion of the amplification product (for example, of one or more barcodes contained in the amplification product) and/or one or more of the polynucleotides with or without amplification, for instance any barcodes contained therein. In some aspects, the provided methods involve performing one or more of the steps described herein, simultaneously and/or sequentially. In some aspects, the steps provided herein may be performed in any suitable order.

In some aspects, the methods provided herein comprise use of one or more polynucleotides, e.g., probe or probes that comprise a split hybridization region comprising a first hybridization region at the 5′ end of a probe that hybridizes to a first portion of the target region in the target nucleic acid and a second hybridization region at the 3′ end of the same probe (or the 3′ end of another probe that forms a probe set with the probe) that hybridizes to a second portion of the target region in the target nucleic acid. In some aspects, upon hybridization of the probe or probe set to the target nucleic acid, two ends of the nucleic acid molecule(s) are positioned by the hybridization to allow for ligation. For example, the two ends of a single circularizable probe molecule or the two ends of a first probe and a second probe can be ligated. In some embodiments, the two ends are positioned adjacently to allow for ligation without gap filling. In some embodiments, the two ends are positioned to allow for ligation with gap filling prior to the ligation.

In some embodiments, a circularizable probe herein comprises from the 3′ to 5′ direction: a first hybridization region, a barcode, and a second hybridization region. In some embodiments, a circularizable probe herein comprises from the 5′ to 3′ direction: a second hybridization region, a barcode, and a first hybridization region. The first and second hybridization regions can form a split hybridization region, and upon probe hybridization to a target nucleic acid, the first and second hybridization regions each comprises an end that is juxtaposed for ligation to each other (with or without gap filling prior to the ligation) to circularize the circularizable probe.

In some embodiments, a probe set comprises the following in both the first probe and the second probe: a split hybridization region and one or more barcodes on an overhang region that does not hybridize to the target nucleic acid.

In some embodiments, there is provided a first probe or probe set (e.g., a first circularizable probe or probe set or a first linear probe or probe set) for detecting a first target nucleic acid and a second probe or probe set (e.g., a second circularizable probe or probe set or a second linear probe or probe set) for detecting a different, such as second, target nucleic acid. In some embodiments, the first probe or probe set is a first circularizable probe or probe set. In some embodiments, the first probe or probe set is a first linear probe or probe set. In some embodiments, the second probe or probe set is a second circularizable probe or probe set. In some embodiments, the second probe or probe set is a second linear probe or probe set. For example, a first target nucleic acid and second target nucleic acid are two different RNA transcripts.

### (i) Kinase Treatment

In some embodiments, after the probe or probe set is hybridized to the target nucleic acid, the sample is contacted with a kinase to phosphorylate one or more ends of the probe or probe set. In some embodiments, the method comprises contacting the biological sample with a polynucleotide kinase (PNK). In some embodiments, the sample is contacted with at least any of 1 U, 5 U, 10 U, 50 U, 100 U, 200 U, 300 U, 400 U, 500 U, 600 U, 700 U, 800 U, 900 U, or 1000 U of a kinase. In some embodiments, at least any of 1 U/μL, 2 U/μL, 3 U/μL, 4 U/μL, 5 U/μL, 6 U/μL, 7 U/μL, 8 U/μL, 9 U/μL, 10 U/μL, 20 U/μL, 30 U/μL, 40 U/μL, or 50 U/μL of a polynucleotide kinase as the final concentration is used to treat a sample comprising cells or tissues. One unit of PNK activity is defined as the amount of enzyme (measured in units, U) that will catalyze the transfer of 1 nmol of phosphate from ATP to the 5′-OH end of a polynucleotide in 30 minutes at an optimum temperature for the enzyme, usually 37° C. In some embodiments, the PNK transfers the gamma phosphate group from adenosine triphosphate (ATP) to the 5′ hydroxyl termini of DNA or RNA (e.g., probes described herein). In some embodiments, the kinase is an enzyme from the family of transferases that transfer phosphorus-containing groups to alcohol groups (phosphotransferases). Systematically, the kinase class may be from the enzyme class known as ATP: 5′-diphospho polynucleotide 5′-phosphotransferase. In some embodiments, the kinase is a T4 PNK or a variant thereof.

In some embodiments, phosphorylation of the one or more ends of the probe or probe set comprises incubating the sample with the kinase (e.g., polynucleotide kinase (PNK)). In some embodiments, the method comprises incubating the sample with the kinase for at least 20 minutes, at least 30 minutes, at least 35 minutes, at least 40 minutes, at least 45 minutes, at least 50 minutes, at least 60 minutes, at least 80 minutes, at least 100 minutes, or at least 120 minutes. In some embodiments, the incubation with the kinase is performed for 20-60 minutes, 20-45 minutes, 20-120 minutes, 30-120 minutes, 30-60 minutes, or 30-90 minutes. In some embodiments, the incubation with kinase is performed at 30° C. to 40° C., e.g., at 37° C.

In some embodiments, the probe or probe set described herein, comprises a non-ligatable end. In some instances, the kinase treatment converts one or more non-ligatable ends to a ligatable end with a 5′ phosphate. In some embodiments, a kinase treatment step is before and/or after one or more wash steps. In some embodiments, a kinase treatment step is before one or more wash steps. In some embodiments, a kinase treatment step is after one or more wash steps. In some embodiments, the wash step comprising removes unbound probes not hybridized to the target nucleic acid from the sample. In some embodiments, the method comprises performing a wash to remove unbound probe or probe sets prior to contacting the cell or tissue sample with the kinase. For example, the wash may be performed using a ligation buffer comprising a salt and ATP. In some cases, the wash is performed with a buffer that is between about pH 7.5 to about pH 8.5. In some cases, the wash is performed with a buffer that is at about pH 8.0. In some embodiments, the sample is washed prior to kinase treatment with a ligation buffer. In some embodiments, the wash buffer comprises MgCl₂, KCl, ATP, DTT, glycerol and/or BSA. In some embodiments, the wash buffer comprises MgCl₂, KCl, ATP, DTT, glycerol and BSA.

In some embodiments, the kinase is provided in excess to phosphorylate the one or more ends of the plurality of probes or probe sets hybridized in the sample. In some embodiments, the kinase is not endogenous to the sample, e.g., the kinase is exogenously provided to the sample where the probes or probe sets are hybridized. In some embodiments, the kinase is not tethered to any molecules of the probes or probe sets (e.g., the sample is not contacted with PNK-labeled nucleic acid probes).

### (ii) Probes

In some embodiments, provided herein is a method for analyzing a biological sample, comprising contacting the biological sample with a circularizable probe or probe set. In some embodiments, provided herein is a method for analyzing a biological sample, comprising contacting the biological sample with a probe set comprising at least two molecules. In some embodiments, the probe set comprises a first probe and a second probe. In some embodiments, the circularizable probe or probe set comprises a linear oligonucleotide sequence that, upon hybridization to a target nucleic acid, such as an RNA molecule, forms a probe that can be circularized. The probe can be circularized via ligation (optionally with primer extension prior to ligation) using the target RNA and/or a DNA splint as a template. In some embodiments, the probe set comprise a first probe and a second probe, and each probe is a linear oligonucleotide sequence that, upon hybridization of the first probe and second probe to a target nucleic acid, such as an RNA molecule, the probes are positioned to be ligated to form a ligated probe molecule. In some embodiments, one or both of the first and second probes comprise an overhang region. In some embodiments, the overhang region is a region that does not hybridize to the target nucleic acid. In some cases, the overhang region may comprise one or more barcode sequences. In some embodiments, at least two ligations are performed join the first and second probes to form a circular ligation product (e.g., a circularized probe). In some embodiments, one ligation is performed to join the first and second probes at one end of each molecule to form a linear ligated probe product. In some embodiments, the probe is adapted for various different probe ligation and detection methods.

In some aspects, the probe comprises a hybridization region that hybridizes to the target region in the target nucleic acid. In some aspects, the hybridization region is a split hybridization region comprising a first hybridization region at the 5′ end of the linear probe molecule and a second hybridization region at the 3′ end of the linear probe molecule. Once hybridized to the complementary sequences in the target nucleic acid, the first and second hybridization regions are positioned in proximity to each other, e.g., is no more than about 5, 4, 3, 2, or 1 nucleotides away. In some embodiments, the first and second hybridization regions are positioned directly adjacent to one another, such that they are configured to be ligated without gap filling or extension prior to ligation. In some embodiments, once hybridized, one end of a probe is ligated to the adjacent ligatable end of the same probe (or of a second probe molecule if using a probe set) to form a circularized probe or a ligated linear probe.

In some embodiments, a probe or probe set comprises one, two, three, four, or more ribonucleotides. In some embodiments, a circularizable probe disclosed herein comprises one, two, three, four, or more ribonucleotides in a DNA backbone. In some embodiments, the one or more ribonucleotides are at and/or near a ligatable 3′ end of the circularizable probe or probe set. The circularizable probe may comprise an optional 3′ RNA base. In some embodiments, a linear probe set (e.g., first and second probes) comprises one, two, three, four, or more ribonucleotides. In some embodiments, a probe set disclosed herein comprises one, two, three, four, or more ribonucleotides in a DNA backbone. In some embodiments, the one or more ribonucleotides are at and/or near a ligatable 3′ end of a probe (e.g., first probe) of the probe set. The probe may comprise an optional 3′ RNA base.

Any suitable circularizable probe or probe set may be used to generate the RCA template which is used to generate the RCA product. In some embodiments, a circularizable probe is in the form of a linear molecule having ligatable ends which may be circularized by ligating the ends together directly or indirectly. For example, in some embodiments, the ends are ligated to each other, or to the respective ends of an intervening (“gap”) oligonucleotide or to an extended 3′ end of the circularizable probe. A circularizable probe may also be provided in two or more parts, namely two or more molecules (e.g., oligonucleotides) which may be ligated together to form a circle. When said RCA template is circularizable it is circularized by ligation prior to RCA. Ligation may be templated using a ligation template, and in the case of probes such as padlock and molecular inversion probes the target analyte may provide the ligation template, and/or the ligation template may be separately provided. In some embodiments, the circularizable RCA template (or template part or portion) will comprise at its respective 3′ and 5′ ends regions of complementarity to corresponding cognate complementary regions (or binding sites) in the ligation template, which may be adjacent where the ends are directly ligated to each other, or non-adjacent, with an intervening “gap” sequence, where indirect ligation is to take place.

(iii) Ligation and Amplification

In some aspects, after contacting the cell or tissue sample with a kinase to phosphorylate one or more ends of the probe or probe set, such as the probe or probe set hybridized to the target nucleic acid, the method further comprises one or more steps such as ligation, extension and/or amplification of the probe or probe set hybridized to the target nucleic acid. In some embodiments, the methods of the present disclosure include the step of performing rolling circle amplification (RCA) in the presence of a target nucleic acid of interest.

In some embodiments, the method comprises using a circularizable construct hybridized to the target nucleic acid comprising the region of interest to generate a product (e.g., comprising a sequence associated with the target nucleic acid). In some aspects, the product is generated using RCA. In some embodiments, the method comprises ligating the ends of a circularizable probe hybridized to the target RNA to form a circularized probe. In some embodiments, the method further comprises generating a rolling circle amplification product (RCP) of the circularized probe. In some embodiments, the RCA comprises a linear RCA. In some embodiments, the RCA comprises a branched RCA. In some embodiments, the RCA comprises a dendritic RCA. In some embodiments, the RCA comprises any combination of the foregoing. In some embodiments, the method further comprises detecting a signal associated with the rolling circle amplification product in the biological sample. In some embodiments, a ligation product of a first and second probe is generated. In some embodiments the ligation product is a circularized probe. In some aspects, the ligation product or a derivative thereof (e.g., extension product) can be detected. In some embodiments, RCA is not performed.

In some embodiments, the circular construct (e.g. circularized probe) is formed using ligation. In some embodiments, the circular construct is formed using template primer extension followed by ligation. In some embodiments, the ligated probe is generated using the target nucleic acid as template. In some embodiments, the circular construct is formed by providing an insert between ends to be ligated. In some embodiments, the circular construct is formed using a combination of any one of the foregoing. In some embodiments, the ligation is a DNA-DNA templated ligation. In some embodiments, the ligation is an RNA-RNA templated ligation. In some embodiments, the ligation is a RNA-DNA templated ligation, for instance ligation of DNA probes is dependent on the RNA template. In some embodiments, a splint (e.g. a splint nucleic acid) is provided as a template for ligation. In some embodiments, the ligation of the probe set (e.g., first and second probe molecules) comprises two or more ligations. In some cases, the two or more ligations comprises a RNA templated ligation and a DNA templated ligation. For example, in some embodiments, the two or more ligations are performed using different ligases.

In some embodiments, a probe set contacted with the sample comprises a first probe and a second probe, wherein the hybridization region of the probe set comprises a split hybridization region comprising a first hybridization region comprised by the first probe of the probe set and a second hybridization region comprised by the second probe of the probe set. In some aspects, the sample is contacted with a splint nucleic acid prior to ligating the probe or probe set. For example, the splint nucleic acid can be contacted with the cell or tissue sample after contacting the target nucleic acid with the probe or probe set. In some cases, the ligation of the probe set comprises two or more ligations. For example, the two or more ligations can comprise a RNA templated ligation and a DNA templated ligation. In some aspects, the two or more ligations can be performed using different ligases. In some cases, the two or more ligations are performed separately and sequentially. In some cases, the two or more ligations can be performed simultaneously. In some embodiments, the two or more ligations comprise a first ligation for ligating a 5′ end of first probe to a 3′ of second probe templated by the target nucleic acid and a second ligation for ligating a 3′ end of the first probe to a 5′ end of the second probe templated by the splint nucleic acid. In some examples, the RNA templated ligation using the target nucleic acid is performed prior to the DNA templated ligation using the splint nucleic acid.

In some embodiments, a circular construct (e.g. circular probe) is directly hybridized to the target nucleic acid. In some embodiments, the circular construct is formed from a modified circularizable probe or probe set. In some embodiments, the circular construct is formed from a probe or probe set capable of DNA-templated ligation. Sec, e.g., U.S. Pat. No. 8,551,710, which is hereby incorporated by reference in its entirety. In some embodiments, the circular construct is formed from a DNA probe or probe set capable of hybridizing to an RNA target nucleic acid (e.g., RNA-templated ligation). Exemplary probes and methods are described in US 2020/022424 which is incorporated herein by reference in its entirety.

The nature of the ligation reaction depends on the structural components of the polynucleotides used to form the circularizable probe. In some embodiments, the probe or probe set after hybridization to the target nucleic acid is positioned to be ready for ligation because no gaps exist between the split hybridization regions, or is ready for a fill-in (e.g. gap-fill) process, which will then permit the ligation of the polynucleotides to form the circularizable probe.

In some embodiments, a 3′ end and a 5′ end of the circularizable probe or probe set are ligated using the target nucleic acid (e.g., RNA) as a template. In some embodiments, the 3′ end and the 5′ end are ligated without gap filling prior to ligation. In some embodiments, the ligation of the 3′ end and the 5′ end is preceded by gap filling. The gap may be 1, 2, 3, 4, 5, or more nucleotides.

In some embodiments, ligating the first ligatable end to a second ligatable end in the probe or probe set comprises enzymatic ligation, chemical ligation, template dependent ligation, and/or template independent ligation. In some embodiments, ligating the first ligatable end to a second ligatable end in the probe or probe set is a template dependent ligation, for example, wherein the ligation depends on hybridization of an interrogatory region to a region of interest in the target nucleic acid. In some embodiments, the ligation comprises using a ligase having an RNA-templated DNA ligase activity and/or an RNA-templated RNA ligase activity. In some embodiments, the enzymatic ligation involves use of a ligase (e.g., an RNA ligase, a DNA ligase). Ligases comprise ATP-dependent double-strand polynucleotide ligases, NAD-i-dependent double-strand DNA or RNA ligases and single-strand polynucleotide ligases, for example any one of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligation comprises using a ligase selected from the group consisting of a Chlorella virus DNA ligase (PBCV DNA ligase), a T4 RNA ligase, a T4 DNA ligase, and a single-stranded DNA (ssDNA) ligase. In some embodiments, the ligation comprises using a PBCV-1 DNA ligase or variant or derivative thereof and/or a T4 RNA ligase 2 (T4 Rn12) or variant or derivative thereof. In some embodiments, the ligase is a T4 RNA ligase. In some embodiments, the ligase comprises a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, a ligation buffer disclosed herein comprises ATP and/or NAD+ as an alternative adenylation donor for nucleic acid ends ligation catalyzed by a ligase.

In some embodiments, the ligation is a direct ligation. In some embodiments, the ligation is an indirect ligation. In some embodiments, a direct ligation occurs between ends of polynucleotides that hybridize immediately adjacently to one another to form a substrate for a ligase enzyme resulting in their ligation to each other (intramolecular ligation). In some embodiments, an indirect ligation is performed when the ends of the polynucleotides hybridize non-adjacently to one another. For instance, non-adjacently hybridized polynucleotides can be separated by one or more intervening nucleotides or gaps. In some embodiments, said ends are not ligated directly to each other, but instead are ligated either via the intermediacy of one or more intervening (so-called gap or gap-filling (oligo) nucleotides) or by the extension of the 3′ end of a probe to fill the “gap” corresponding to said intervening nucleotides (intermolecular ligation). In some cases, the gap of one or more nucleotides between the hybridized ends of the polynucleotides may be filled by one or more gap (oligo) nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In some embodiments, the gap is a gap of about 1, 2, 3, 4, 5, 6, 7, 8, 9 or 10 or more nucleotides, or of any integer (or range of integers) of nucleotides in between any of the indicated values. In some embodiments, the gap between said terminal regions is filled by a gap oligonucleotide or by extending the 3′ end of a polynucleotide. In some cases, ligation involves ligating the ends of the probe to at least one gap (oligo) nucleotide, such that the gap (oligo) nucleotide becomes incorporated into the resulting polynucleotide. In some embodiments, the ligation herein is preceded by gap filling. In some embodiments, the ligation herein does not require gap filling.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of hybridized mismatched ligation substrates (e.g., wherein the interrogatory region is not complementary to the region of interest, such that the probe or probes are expected to have a slightly lower Tₘ around the mismatch) over hybridized fully base-paired ligation substrates (e.g., wherein the interrogatory region is complementary to the region of interest). Thus, high-fidelity ligation can be achieved through a combination of the intrinsic selectivity of the ligase active site and balanced conditions to reduce the incidence of annealed mismatched dsDNA.

In some embodiments, the method further comprises prior to ligating the first ligatable end to a second ligatable end in the probe or probe set, a step of removing molecules of the probe or probe set that are not bound to the target nucleic acid from the biological sample.

Following formation of, e.g., the circularized probe, in some instances, an amplification primer is added. In other instances, the amplification primer is added with the circularizable probes. In some embodiments, the amplification primer is added after contacting the sample with the kinase. In some instances, the amplification primer also is complementary to the target nucleic acid and the circularizable probe. In some embodiments, an amplification primer is not needed and an extension reaction uses the target nucleic acid as a primer. In some embodiments, a washing step is performed to remove any unbound probes, primers, and/or other components. In some embodiments, the wash is a stringency wash. Washing steps can be performed at any point during the process to remove, for example, non-specifically bound probes, probes that have not ligated, and/or other components. In some embodiments, the stringency is increased in the hybridization of the probe or probe set to the target nucleic acid, reducing or negating the need of performing a stringency wash.

In some embodiments, upon addition of a DNA polymerase in the presence of appropriate dNTP precursors and other cofactors, the amplification primer is elongated by replication of multiple copies of the circularized probe template (e.g., a concatemer of the template is generated). This amplification product (e.g. RCP) can be detected using, e.g., the secondary and higher order probes and detection oligonucleotides described herein. In some embodiments, the sequence of the amplicon (e.g. RCP) or a portion thereof, is determined or otherwise analyzed, for example by using detectably labeled probes and imaging. The sequencing or analysis of the amplification products can comprise sequencing by hybridization, sequencing by ligation, and/or fluorescent in situ sequencing, and/or wherein the in situ hybridization comprises sequential fluorescent in situ hybridization. In some instances, sequencing using, e.g., the secondary and higher order probes and detection oligonucleotides described herein.

In some embodiments, the method further comprises generating the product of the circularized probe (e.g. the RCP) in situ in the biological sample. In some embodiments, the product is generated using rolling circle amplification (RCA). In some embodiments, the RCA comprises a linear RCA, a branched RCA, a dendritic RCA, or any combination thereof. In some embodiments, the product is generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (such as an amplicon) containing multiple copies of the cDNA. Techniques for rolling circle amplification (RCA) include but are not limited to linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49 (11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29: el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801, each of which is incorporated herein in its entirety). Exemplary polymerases for use in RCA comprise DNA polymerase such phi29 (q29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) are modified to contain functional groups that can be used as anchoring sites to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465, all of which are incorporated herein in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, an exemplary workflow for analyzing a biological sample, comprises contacting the biological sample with a circularizable probe, wherein: the probe comprises a first hybridization region capable of hybridizing to a first target sequence in a target nucleic acid in the biological sample, a second hybridization region capable of hybridizing to a second target sequence in the target nucleic acid, wherein upon hybridization of the circularizable probe to the target nucleic acid, the probe is treated with a kinase to phosphorylate the 5′ end of the probe; ligating the phosphorylated 5′ end to the 3′ end of the circularizable probe to generate a circularized probe using the target nucleic acid as template; amplifying the circularized probe using rolling circle amplification (RCA) to generate an RCA product; and detecting the RCA product in the biological sample.

In some embodiments, an example of a workflow for analyzing a biological sample comprises contacting a target RNA with a first probe and a second probe simultaneously or in any order, wherein the first probe comprises a first hybridization region capable of hybridizing to a first target RNA sequence in the target RNA, and upon hybridization of the first and second probes to the target RNA, the sample is contacted with a kinase to phosphorylate the 5′ ends of the first and second probes; ligating the phosphorylated 5′ end(s) of one probe to the 3′ end(s) of the other probe to generate a ligated probe; and detecting the ligated probe or a product thereof. In some embodiments, one ligation is performed using the target RNA as template. In some embodiments, the ligated probe is linear. In some embodiments, a second ligation is performed using a splint nucleic acid (e.g., DNA splint). In some embodiments, the ligated probe is circularized. In some embodiments, target RNA is in a cell or tissue sample and the ligated probe and/or the product thereof is generated in the cell or tissue sample. In some embodiments, the ligated probe and/or the product thereof is detected in the cell or tissue sample.

In some embodiments, an example of a workflow for analyzing a biological sample comprises contacting a target RNA with a first probe and a second probe simultaneously or in any order, wherein the first probe comprises a first hybridization region capable of hybridizing to a first target RNA sequence in the target RNA, and the second probe comprises a second hybridization region capable of hybridizing to a second target RNA sequence in the target RNA, and upon hybridization of the first and second probes to the target RNA, the sample is contacted with a kinase to phosphorylate the 5′ ends of the first and second probes; ligating the phosphorylated 5′ ends of the first and second probes to the 3′ ends of the second and first probes, respectively, to generate a ligated probe; and detecting the ligated probe or a product thereof.

### (iv) Analytes

The methods and compositions disclosed herein can be used to detect and analyze a wide variety of different analytes. In some embodiments, the methods and compositions are for analysis of a plurality of different and endogenous analytes (and/or products thereof generated in situ) present in a cell or a biological sample, such as a tissue sample. In some aspects, an analyte can include any biological substance, structure, moiety, or component to be analyzed. In some aspects, a target analyte (e.g., a target nucleic acid) disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (a) Endogenous Analytes

In some embodiments, an analyte herein is endogenous to a biological sample and can include nucleic acid analytes and/or non-nucleic acid analytes. Methods and compositions disclosed herein can be used to analyze nucleic acid analytes (e.g., using a nucleic acid probe or probe set as described in Section II. (ii) that directly or indirectly hybridizes to a target nucleic acid) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte is an organelle. For example, organelles might be nuclei or mitochondria. In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte is a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In some embodiments, an analyte is extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In some embodiments, the analyte comprises a target sequence. In some embodiments, the target sequence is endogenous to the sample, generated in the sample, added to the sample, or associated with an analyte in the sample. In some embodiments, the target sequence is a single-stranded target sequence. For example, the single-stranded target sequence can be a sequence in a rolling circle amplification product. In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

### (b) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent includes an agent that interacts with an analyte. In some embodiments, the analyte is an endogenous analyte in a sample. In some embodiments, the labelling agents comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences. In some embodiments, the one or more barcode sequences comprise a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any one of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, all of which are herein incorporated by reference in their entireties.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein. For example, the protein can be a protein on a surface of the biological sample (e.g., a cell) or an intracellular protein. In some embodiments, a plurality of analyte labelling agents comprising a plurality of analyte binding moieties bind a plurality of analytes present in a biological sample. In some embodiments, the plurality of analytes includes a single species of analyte. In some embodiments, the single species of analyte is a single species of polypeptide. In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different. For example, members of the plurality of analyte labelling agents can have two or more species of analyte binding moieties, wherein each of the two or more species of analyte binding moieties binds a single species of analyte, e.g., at different binding sites. In some embodiments, the plurality of analytes includes multiple different species of analyte. In some embodiments, the multiple different species of analytes are multiple different species of polypeptides.

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any one of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31 (2): 708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer binding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample are subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### (c) Target Sequences

In some embodiments, provided herein are methods and compositions for analyzing endogenous analytes (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof. Endogenous analytes can include, for example, viral or cellular DNA or RNA. In some embodiments, the product is a hybridization product, a ligation product, an extension product, a replication product, a transcription/reverse transcription product, and/or an amplification product. In some embodiments, a labelling agent that directly or indirectly binds to an analyte in the biological sample is analyzed. In some embodiments, a product of a labelling agent that directly or indirectly binds to an analyte in the biological sample is analyzed. In some embodiments, the product of the labelling agent is a hybridization product, a ligation product, an extension product, a replication product, a transcription/reverse transcription product, and/or an amplification product. In some embodiments, the extension product is formed by a DNA polymerase or RNA polymerase. In some embodiments, the amplification product is a rolling circle amplification (RCA) product. A target sequence for a probe disclosed herein may be comprised in any analyte disclose herein, including an endogenous analyte (e.g., a viral or cellular nucleic acid), a labelling agent, or a product of an endogenous analyte and/or a labelling agent.

### III. Spatial Array Capture

In some embodiments, after the kinase treatment and ligation of the probe or probe sets described in Section II, the assay further comprises one or more optional steps for transferring the probes (or a product or derivative thereof) to a substrate e.g., an array. In some embodiments, the array is a substrate with a plurality of immobilized spatially barcoded oligonucleotides. In some embodiments, the probes (e.g., first probe and second probe) can be ligated and then transferred to an array. In some embodiments, the probes (e.g., first probe and second probe) can be ligated, released from the cell or tissue sample, and captured on an array. In some embodiments, a product (e.g., extension product) or derivative of the ligated probes (e.g., ligated first-second probe) can be transferred to an array. In some embodiments, the product is an extension product. In some embodiments, the method comprises hybridizing a probe set comprising a first probe and a second probe to a target nucleic acid in a biological sample, wherein the probe set lacks a 5′ phosphate, treating the biological sample with a kinase to phosphorylate a 5′ end of the hybridized probe set, ligating the first and second probe of the hybridized probe set to generate a linear ligation product, and transferring the linear ligation product to the array. In some embodiments, the method comprises hybridizing a circularizable probe or probe set lacking a 5′ phosphate to a target nucleic acid in a biological sample, treating the biological sample with a kinase to phosphorylate a 5′ end of the hybridized circularizable probe or probe set, ligating ends of the hybridized circularizable probe or probe set to generate a circularized probe, and transferring the circularized probe to the array.

In some embodiments, provided herein is a method for analyzing a target nucleic acid in a cell or tissue sample, comprising contacting the cell or tissue sample with a plurality of probe sets, wherein each probe set of the plurality of probe sets comprises a first probe and a second probe, wherein the target nucleic acid comprises a first target region and a second target region and the first probe and second probe hybridize to the first and second target regions, respectively; contacting the cell or tissue sample with a kinase to phosphorylate the 5′ ends of the first probes and/or the second probes hybridized to the target nucleic acid; and ligating the first probes to the second probes hybridized to the same target nucleic acid to form a plurality of ligated probes in the cell or tissue sample; and detecting the plurality of ligated probes or a product thereof. In some embodiments, the ligated probes are generated from ligating the 3′ end of the first probe to the phosphorylated 5′ end of the second probe. In some embodiments, the ligated probes are linear probes (i.e. not circularized). In some embodiments, the plurality of ligated probes or a product thereof is detected on an array or by nucleic acid sequencing. In some embodiments, the method comprises generating a spatially barcoded oligonucleotide comprising (i) a sequence of the ligated probe or product thereof or complement thereof and (ii) a sequence of the spatial barcode sequence or complement thereof and releasing the spatially barcoded oligonucleotide from the substrate (e.g., array) for sequencing. In some embodiments, the substrate is an array. For example, the ligated probe (or a derivative thereof) can be captured by an oligonucleotide (e.g., a capture probe immobilized, directly or indirectly, on a substrate) comprising a capture sequence complementary to a sequence of the ligated probe(s) on an array, optionally amplified, and sequenced, thus determining the location and optionally the abundance of the analyte in the biological sample.

Array-based spatial analysis methods involve the transfer of one or more analytes (such as ligated probes described herein) from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array. Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604, 182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2013/171621, WO 2018/091676, WO 2020/176788, Rodriques et al., Science 363 (6434): 1463-1467, 2019; Lee et al., Nat. Protoc. 10 (3): 442-458, 2015; Trejo et al., PLOS ONE 14 (2): e0212031, 2019; Chen et al., Science 348 (6233): aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination; all of which are incorporated herein in their entireties. Further non-limiting aspects of spatial analysis methodologies and compositions are described herein.

In some cases, spatial analysis can be performed by attaching and/or introducing a molecule having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. Some such methods of spatial analysis are described in Section (III) of WO 2020/176788 and/or U.S. Patent Application Publication No. 2020/0277663.

In some cases, spatial analysis can be performed by detecting multiple oligonucleotides (e.g., ligated probe sets as described herein) that hybridize to a target nucleic acid. In some instances, for example, spatial analysis can be performed by hybridization of two oligonucleotides (e.g., a first probe and a second probe) to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end, and at least one of the oligonucleotides lacks a 5′ phosphate. In some instances, the polynucleotides (e.g., (e.g., a circularizable probe or probe set)) includes a capture domain (e.g., a poly (A) sequence, a non-homopolymeric sequence. In some embodiments, after hybridization to the analyte, and kinase treatment, a ligase (e.g., SplintR ligase) ligates the two oligonucleotides (e.g., of a probe set) together, creating a ligation product (e.g., a ligated probe set that forms a single linear molecule). In some instances, the two or more molecules of the probe set hybridize to sequences that are not directly adjacent to one another. For example, in some embodiments, hybridization of the two probes of a probe set creates a gap between the hybridized nucleic acid molecules. In some instances, a polymerase (e.g., a DNA polymerase) can extend one of the nucleic acid molecules prior to ligation. After ligation, the ligation product is released from the analyte. In some instances, the ligation product is released using an endonuclease (e.g., RNAse H). The released ligation product (or a derivative thereof) can then be captured by an oligonucleotide (e.g., a capture probe immobilized, directly or indirectly, on a substrate) comprising a capture sequence complementary to a sequence of the ligated probe(s) on an array, optionally amplified, and sequenced, thus determining the location and optionally the abundance of the analyte in the biological sample. In some embodiments, the ligated probe(s) comprise a complementary capture sequence. In some instances, a ligated probe comprises an overhang region (e.g., a region that does not hybridize to the target nucleic acid) comprising the complementary capture sequence. In some embodiments, the overhang region is a region that does not hybridize to the target nucleic acid. In some embodiments, the capture sequence comprises a polyA sequence. In some embodiments, the oligonucleotide (e.g., capture probe) comprising the capture sequence comprises a spatial barcode sequence. In some embodiments, the oligonucleotide is a capture probe. In some embodiments, the method comprises generating a spatially barcoded oligonucleotide comprising (i) a sequence of the ligated probe or product thereof or complement thereof and (ii) a sequence of the spatial barcode sequence or complement thereof.

In some embodiments, after ligating the first and second probe of the probe set, the method comprises a step of removing unligated molecules of the probe set. For example, first and/or second probes that are not ligated can be removed. In some embodiments, ligation of the probe set (e.g., the first and second probe) stabilizes hybridization of the probes to the target nucleic acid. In some embodiments, the method comprises one or more stringency washes to remove unligated probes. In some embodiments, the one or more stringency washes are performed prior to migrating probes to capture oligonucleotides.

A capture probe can be any molecule capable of capturing (directly or indirectly) a linear probe, probe set, or product thereof corresponding to an analyte in a biological sample, or a circularizable probe, probe set or product thereof corresponding to an analyte in a biological sample. In some embodiments, the capture probe is a nucleic acid or a polypeptide. In some embodiments, the capture probe includes a barcode. In some embodiments, the barcode comprises a spatial barcode and/or a unique molecular identifier (UMI)) and a capture domain. In some embodiments, a capture probe includes a cleavage domain and/or a functional domain. In some embodiments, the functional domain is a primer-binding site. In some embodiments, the primer-binding site is for next-generation sequencing (NGS). Sec, e.g., Section (II) (b) (e.g., subsections (i)-(vi)) of WO 2020/176788 and/or U.S. Patent Application Publication No. 2020/0277663. Generation of capture probes can be achieved by any appropriate method, including those described in Section (II) (d) (ii) of WO 2020/176788 and/or U.S. Patent Application Publication No. 2020/0277663.

In some embodiments, at least one of the probes for binding a target nucleic acid (e.g., probe set comprising a first and second probe) comprises a sequence complementary to a sequence comprised by an oligonucleotide comprising a capture sequence (e.g., capture probe). Probes or probe sets can include those as described in Section II. In some embodiments, the oligonucleotide is a capture probe. In some embodiments, a plurality of the probes (e.g., first or second probes, or ligated probes generated from the first and second probes) comprise a common sequence for hybridizing to a capture sequence on the oligonucleotide (e.g. capture probe). In some embodiments, the plurality of probes is first or second probes, or ligated probes generated from the first and second probes. In some embodiments, the capture probes are spatially barcoded capture probes attached to a substrate (e.g., array). In some embodiments, the substrate is an array. In some instances, the spatially barcoded capture probes described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer binding sequence (such as an R1, R2, or partial R1 or R2 sequence). In some embodiments, the method comprises generating a spatially barcoded oligonucleotide comprising (i) a sequence of the ligated probe or product thereof or complement thereof and (ii) a sequence of the spatial barcode sequence or complement thereof. In some examples, a ligation product (or a derivative thereof) can be captured by the oligonucleotides comprising the capture sequence (e.g. capture probes) and associated with a spatial barcode, optionally amplified, and sequenced, thus determining the location of the target nucleic acid. In some cases, the spatially barcoded oligonucleotides can be attached to functional sequences described herein (such as sequence specific flow cell attachment sequences) prior to analysis. In some cases, the spatially barcoded oligonucleotide (or a product or derivative thereof) can be released from the substrate (e.g., array) prior to analysis. In some cases, the spatially barcoded oligonucleotide can be further processed and subjected to one or more reactions prior to analysis (e.g., extension, amplification, or other reactions). In some embodiments, the spatially barcoded oligonucleotides are detected by nucleic acid sequencing. In some other embodiments, the spatially barcoded oligonucleotides are detected at the spatial location in the biological sample.

There are methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially barcoded array (e.g., including spatially barcoded capture probes). Another method is to cleave spatially barcoded capture probes from an array and promote the spatially barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II) (b) (vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

In some embodiments, an extended capture probe is a capture probe having additional nucleotides added to the terminus (e.g., 3′ or 5′ end) of the capture probe thereby extending the overall length of the capture probe. For example, an extended 3′ end can comprise additional nucleotides added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte (such as a ligated probe set as described above) or intermediate agent specifically bound to the capture domain of the capture probe. In some embodiments, the capture probe is extended using reverse transcription. In some embodiments, the capture probe is extended using one or more DNA polymerases. The extended capture probes can include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, amplification is in bulk solution. In some embodiments, amplification is on the array. In some embodiments, downstream analysis comprises DNA sequencing. In some embodiments, extended capture probes (e.g., DNA molecules) act as templates for an amplification reaction. In some embodiments, the amplification reaction is a polymerase chain reaction.

Additional variants of spatial analysis methods, including in some embodiments, an imaging step, are described in Section (II) (a) of WO 2020/176788 and/or U.S. Patent Application Publication No. 2020/0277663, all of which are incorporated by reference herein in their entireties. Analysis of captured analytes such as ligated probes described herein (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II) (g) of WO 2020/176788 and/or U.S. Patent Application Publication No. 2020/0277663, all of which are incorporated by reference herein in their entireties. Some quality control measures are described in Section (II) (h) of WO 2020/176788 and/or U.S. Patent Application Publication No. 2020/0277663, all of which are incorporated by reference herein in their entireties.

Typically, for spatial array-based methods, a substrate functions as a support for direct or indirect attachment of capture probes to features of the array. A feature can be an entity that acts as a support or repository for various molecular entities used in spatial analysis. In some embodiments, some or all of the features in an array are functionalized for analyte capture. Exemplary substrates are described in Section (II) (c) of WO 2020/176788 and/or U.S. Patent Application Publication No. 2020/0277663, all of which are incorporated by reference herein in their entireties. Exemplary features and geometric attributes of an array can be found in Sections (II) (d) (i), (II) (d) (iii), and (II) (d) (iv) of WO 2020/176788 and/or U.S. Patent Application Publication No. 2020/0277663, all of which are incorporated by reference herein in their entireties.

Generally, analytes and/or corresponding probes (e.g., described in Section II) can be captured when contacting a biological sample with a substrate including capture probes (e.g., a substrate with capture probes embedded, spotted, printed, fabricated on the substrate, or a substrate with features (e.g., wells) comprising capture probes). In the context of spatial array capture, contacting a biological sample with a substrate can comprise any contact (e.g., direct or indirect) such that capture probes can interact (e.g., bind covalently or non-covalently (e.g., hybridize)) with analytes from the biological sample. In some aspects, the oligonucleotide comprises a capture sequence complementary to a sequence of the ligated probe and/or the product thereof. In some embodiments, a target nucleic acid is at a location in a biological sample, the ligated probe is generated at the location in the biological sample, and the ligated probe and/or product thereof is covalently or noncovalently attached to an oligonucleotide (e.g., capture probe) immobilized on a substrate. In some embodiments, a target nucleic acid is covalently or noncovalently attached to an oligonucleotide (e.g., capture probe) immobilized on a substrate and the ligated probe and/or product thereof is generated. In some embodiments, the oligonucleotide is a capture probe. Capture can be achieved actively (e.g., using electrophoresis) or passively (e.g., using diffusion). Analyte capture is further described in Section (II) (c) of WO 2020/176788 and/or U.S. Patent Application Publication No. 2020/0277663.

During analysis of spatial information, sequence information for a spatial barcode associated with an analyte (e.g., ligated first-second probe or product or derivative thereof) is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663, all of which are incorporated by reference herein in their entireties. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020), all of which are incorporated by reference herein in their entireties.

In some embodiments, spatial analysis is performed using dedicated hardware and/or software, such as any of the systems described in Sections (II) (e) (ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320, all of which are incorporated by reference herein in their entireties.

Suitable systems for performing spatial analysis can include components such as a chamber (e.g., a flow cell or scalable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854, all of which are incorporated by reference herein in their entireties.

Prior to transferring analytes (such as ligated probes or products generated therefrom) from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655, US20220062246, and spatial analysis methods are generally described in WO 2020/061108 and/or U.S. patent application Ser. No. 16/951,864, all of which are incorporated by reference herein in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843, all of which are incorporated by reference herein in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

In some embodiments, an exemplary workflow for analyzing a biological sample, comprises contacting the target RNA with a first probe and a second probe simultaneously or in any order, wherein the first probe comprises a first hybridization region capable of hybridizing to a first target RNA sequence in the target RNA, the second probe comprises a second hybridization region capable of hybridizing to a second target RNA sequence in the target RNA, and upon hybridization of the first and second probes to the target RNA, treating the first probe and second probe with a kinase to phosphorylate one or more ends of the probe(s); ligating the first and second probes to generate a ligated probe using the target RNA as template; and detecting the ligated probe or a product thereof. In some embodiments, the target RNA is in a cell or tissue sample and the ligated probe and/or the product thereof is generated in the cell or tissue sample. In some embodiments, the ligated probe and/or the product thereof is covalently or noncovalently attached to an oligonucleotide immobilized on a substrate. In some embodiments, the oligonucleotide comprises a spatial barcode sequence and a spatially barcoded oligonucleotide comprising (i) a sequence of the ligated probe or product thereof or complement thereof and (ii) a sequence of the spatial barcode sequence or complement thereof is generated and sequenced.

### IV. Detection and Analysis

In some aspects, the provided methods are employed for in situ analysis of target nucleic acids, for example for in situ sequencing or multiplexed analysis in intact tissues or a sample with preserved cellular or tissue structure. In some aspects, the provided methods can be used to detect or determine the identity or amount in situ of a target nucleic acid of interest. In some aspects, the provided methods are employed for analysis of target nucleic acids using a spatial array, as discussed, for example in Section III.

In some aspects, after formation of a hybridization complex comprising nucleic acid probes and/or probe sets described in Section II and any one or more further processing steps (e.g., ligation, extension amplification, capture, or any combination thereof), the method can further include detection of the probe or probe set hybridized to the target nucleic acid or any products generated therefrom or a derivative thereof. In some embodiments, the method further comprises imaging the biological sample to detect a ligation product or a circularized probe or product thereof. In some embodiments, a sequence of the ligation product, rolling circle amplification product, or other generated product is analyzed in situ in the biological sample. In some embodiments, the imaging comprises detecting a signal associated with a fluorescently labeled probe that directly or indirectly binds to a rolling circle amplification product of the circularized probe. In some embodiments, the sequence of the ligation product, rolling circle amplification product, extension product, and/or other generated product is analyzed by sequential hybridization, sequencing by hybridization, sequencing by ligation, sequencing by synthesis, sequencing by binding, or a combination thereof. In some cases, a spatially barcoded analyte (or a product or derivative thereof) can be released from an array prior to analysis.

In some embodiments, a sequence associated with the target nucleic acid or the probe(s) comprises one or more barcode sequences or complements thereof. In some embodiments, the sequence of the rolling circle amplification product comprises one or more barcode sequences or complements thereof. In some embodiments, a ligated linear probe (e.g., generated from a first and second probe described herein) comprises one or more barcode sequences or complements thereof. In some embodiments, the ligated linear probe is generated from a first and second probe described herein. In some embodiments, a ligated linear probe comprises an overhang region (e.g., a region that does not hybridize to the target nucleic acid) comprising one or more barcode sequences or complements thereof, which can be detected according to any of the methods described herein. In some embodiments, the overhang region is a region that does not hybridize to the target nucleic acid. In some embodiments, the detection comprises signal amplification. In some embodiments, the one or more barcode sequences comprises a barcode sequence corresponding to the target nucleic acid. In some embodiments, the one or more barcode sequences can comprise a barcode sequence corresponding to the sequence of interest, such as variant(s) of a single nucleotide of interest.

In some aspects, any of the probe(s) described herein can comprise one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or UMI). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, barcodes or complements thereof (e.g., barcode sequences or complements thereof comprised by the probes disclosed herein or products thereof) can be analyzed (e.g., detected or sequenced) using any suitable method or technique, including those described herein, such as sequencing by synthesis (SBS), sequencing by ligation (SBL), or sequencing by hybridization (SBH). In some embodiments, the barcodes or complements thereof are comprised by the probes disclosed herein or products thereof.

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4\ complexity given a sequencing read of N bases, and a much shorter sequencing read is required for molecular identification compared to non-barcode sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which are an efficient read-out in terms of information per cycle of sequencing. Because the barcode sequences are pre-determined, they can also be designed to feature error detection and correction mechanisms, see, e.g., U.S. Pat. Pub. 20190055594 and 20210164039, all of which are herein incorporated by reference in their entireties.

In some embodiments, the target nucleic acid is at a location in a biological sample and the ligated probe is generated at the location in the biological sample, and the ligated probe and/or the product thereof is detected at the location in the biological sample.

In some embodiments, the detecting comprises contacting the biological sample with one or more detectably-labeled probes that directly or indirectly hybridize to the rolling circle amplification product, and dehybridizing the one or more detectably-labeled probes from the rolling circle amplification product. In some embodiments, the contacting and dehybridizing steps are repeated with the one or more detectably-labeled probes and/or one or more other detectably-labeled probes that directly or indirectly hybridize to the rolling circle amplification product. In some embodiments, the detectably-labeled probes directly hybridize to the rolling circle amplification product (e.g., generated as described in Section II).

In some embodiments, the detecting comprises contacting the biological sample with one or more intermediate probes that directly or indirectly hybridize to the rolling circle amplification product, wherein the one or more intermediate probes are detectable using one or more detectably-labeled probes. In some embodiments, the detecting further comprises dehybridizing the one or more intermediate probes and/or the one or more detectably-labeled probes from the rolling circle amplification product. In some embodiments, the contacting and dehybridizing are repeated with the one or more intermediate probes, the one or more detectably-labeled probes, one or more other intermediate probes, and/or one or more other detectably-labeled probes.

In some embodiments, the method comprises detecting the RCA product by hybridizing one or more linear probes to the RCA product. In some embodiments, a linear probe is one that comprises a target recognition sequence (e.g., a sequence complementary to a barcode sequence or subunit thereof in the RCA product) and a sequence that does not hybridize to a target nucleic acid, such as a 5′ overhang, a 3′ overhang, and/or a linker or spacer (which may comprise a nucleic acid sequence or a non-nucleic acid moiety). In some embodiments, the target recognition sequence is a sequence complementary to a barcode sequence or subunit thereof in the RCA product. In some embodiments, the sequence (e.g., the 5′ overhang, 3′ overhang, and/or linker or spacer) is non-hybridizing to the target nucleic acid but may hybridize to one another and/or one or more other probes, such as detectably labeled probes. In some embodiments, the sequence comprises a 5′ overhang, 3′ overhang, and/or linker or spacer. In some embodiments, a linear probe is one that comprises a target recognition sequence (e.g., a sequence complementary to a barcode sequence or subunit thereof in the RCA product) and an optically detectable label. In some embodiments, the target recognition sequence is a sequence complementary to a barcode sequence or subunit thereof in the RCA product.

In some embodiments, the detection is spatial. In some embodiments, spatial detection is detection in two or three dimensions. In some embodiments, the detection is quantitative, e.g., the amount or concentration of a primary nucleic acid probe (and of a target nucleic acid) or a stem-loop structure may be determined. In some embodiments, the primary probes, secondary probes, higher order probes, and/or detectably labeled probes may comprise any one of a variety of entities able to hybridize a nucleic acid, e.g., DNA, RNA, LNA, and/or PNA, etc., depending on the application.

In some embodiments, disclosed herein is a multiplexed assay where multiple targets (e.g., nucleic acids such as genes or RNA transcripts, or protein targets) are probed with multiple primary probes (e.g., circularizable primary probes), and optionally multiple secondary probes hybridizing to the primary barcodes (or complementary sequences thereof) are all hybridized at once, followed by sequential secondary barcode detection and decoding of the signals. In some embodiments, the multiple targets are nucleic acids such as genes or RNA transcripts, or protein targets. In some embodiments, the multiple primary probes are circularizable primary probes. In some embodiments, detection of barcodes or subsequences of the barcode occurs in a cyclic manner.

In some aspects, the provided methods involve analyzing, e.g., detecting or determining, one or more sequences present in the polynucleotides (e.g., probe or probe set) and/or in a product or derivative thereof, such as in an amplified circularized probe. In some embodiments, the detection comprises providing detection probes, such as probes for performing a chain reaction that forms an amplification product. In some embodiments, the chain reaction is HCR. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region of interest in a target nucleic acid. In some embodiments, the provided methods involve analyzing, e.g., detecting or determining, one or more sequences present in probes or products thereof. In some embodiments, the one or more sequences are barcode sequences or products thereof.

In some embodiments, a method disclosed herein also comprises one or more signal amplification components. In some embodiments, the present disclosure relates to the detection of nucleic acids sequences in situ using probe hybridization and generation of amplified signals associated with the probes, wherein background signal is reduced and sensitivity is increased. In some embodiments, the RCA product generated using a method disclosed herein is detected with a method that comprises signal amplification.

Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594, the content of which is herein incorporated by reference in its entirety), hybridization chain reaction, assembly of topologically catenated DNA structures using serial rounds of chemical ligation (clampFISH), signal amplification via hairpin-mediated concatemerization (e.g., as described in US 2020/0362398, the content of which is herein incorporated by reference in its entirety), e.g., primer exchange reactions such as signal amplification by exchange reaction (SABER) or SABER with DNA-Exchange (Exchange-SABER). In some embodiments, a non-enzymatic signal amplification method may be used.

The detectable reactive molecules may comprise tyramide, such as used in tyramide signal amplification (TSA) or multiplexed catalyzed reporter deposition (CARD)-FISH. In some embodiments, the detectable reactive molecule is releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). In some embodiments, the fluorophore-labeled reactive molecule is tyramide. Exemplary detectable reactive reagents and methods are described in U.S. Pat. No. 6,828,109, US 2019/0376956, US 2019/0376956, WO 2020/102094, US 2022/0026433, WO 2020/163397, US 2022/0128565, and WO 2021/067475, all of which are incorporated herein by reference in their entireties.

In some embodiments, hybridization chain reaction (HCR) is used for signal amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101 (43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28 (11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an initiator nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g., initiator or other HCR monomer) when the monomers are in the hairpin structure may be referred to as the toehold region (or input domain). The first HCR monomers each further comprise a sequence which is complementary to a sequence in the exposed toehold region of the second HCR monomers. This sequence of complementarity in the first HCR monomers may be referred to as the interacting region (or output domain). Similarly, the second HCR monomers each comprise an interacting region (output domain), e.g., a sequence which is complementary to the exposed toehold region (input domain) of the first HCR monomers. In the absence of the HCR initiator, these interacting regions are protected by the secondary structure (e.g., they are not exposed), and thus the hairpin monomers are stable or kinetically trapped (also referred to as metastable), and remain as monomers (e.g., preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g., the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived. Branching HCR systems have also been devised and described (see, e.g., WO 2020/123742 and US 20220064697, all of which are herein incorporated by reference in their entireties), and may be used in the methods herein.

In some embodiments, similar to HCR reactions that use hairpin monomers, linear oligo hybridization chain reaction (LO-HCR) is also used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species do not comprise a hairpin structure. In some embodiments, the plurality of LO-HCR monomers do not comprise a metastable secondary structure. In some embodiments, the LO-HCR polymer do not comprise a branched structure. In some embodiments, performing the linear oligo hybridization chain reaction comprises contacting the target nucleic acid molecule with the initiator to provide the initiator hybridized to the target nucleic acid molecule. In some embodiments, the target nucleic acid molecule and/or the analyte is an RCA product.

In some embodiments, detection of nucleic acids sequences in situ includes combination of RCA with an assembly for branched signal amplification. In some embodiments, the assembly complex comprises an amplifier hybridized directly or indirectly (via one or more oligonucleotides) to a sequence of the RCA product. In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), all of which are herein entirely incorporated by reference in their entireties.

In some embodiments, the RCA product is detected with a method that comprises signal amplification by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a sample comprising RCA products generated using methods described herein. In various embodiments, the RCA product may be contacted with a plurality of concatemer primers and a plurality of labeled probes, see e.g., U.S. Pat. Pub. No. US20190106733, the content of which is herein incorporated by reference in its entirety, for exemplary molecules and PER reaction components.

In some embodiments, the methods comprise sequencing all or a portion of the amplification product, such as one or more barcode sequences present in the amplification product. In some embodiments, extended capture probes are amplified (e.g., in bulk solution or on the array) and analyzed, e.g., via DNA sequencing. In some embodiments, amplification is in bulk solution or on an array. In some embodiments, analysis comprises DNA sequencing.

In some embodiments, the product or derivative of a first and second probe ligated together after hybridizing to the target nucleic acid is analyzed by sequencing. In some embodiments, the analysis and/or sequence determination comprises sequencing all or a portion of the amplification product or the probe(s) and/or in situ hybridization to the amplification product or the probe(s). In some embodiments, the sequencing step involves sequencing by hybridization, sequencing by ligation, and/or fluorescent in situ sequencing, hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction. See e.g., US 2017/0009278, which is the content of which is herein incorporated by reference in its entirety, for exemplary probes and HCR reaction components. In some embodiments, the detection or determination comprises hybridizing to the amplification product a detection oligonucleotide labeled with a fluorophore, an isotope, a mass tag, or a combination thereof. In some embodiments, the detection or determination comprises imaging the amplification product. In some embodiments, the target nucleic acid is an mRNA in a tissue sample, and the detection or determination is performed when the target nucleic acid and/or the amplification product is in situ in the tissue sample.

In some aspects, provided herein are in situ assays using microscopy as a readout, e.g., nucleic acid sequencing, hybridization, or other detection or determination methods involving an optical readout. In some aspects, detection or determination of a sequence of one, two, three, four, five, or more nucleotides of a target nucleic acid is performed in situ in a cell in an intact tissue. In some aspects, the detection or determination is of a sequence associated with or indicative of a target nucleic acid. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product). In some embodiments, the amplification product or portion thereof is an RCA product or portion thereof. In some embodiments, a method for spatially profiling analytes such as the transcriptome or a subset thereof in a biological sample is provided. Methods, compositions, kits, devices, and systems for these in situ assays, comprising spatial genomics and transcriptomics assays, are provided. In some embodiments, a provided method is quantitative and preserves the spatial information within a tissue sample without physically isolating cells or using homogenates. In some embodiments, the present disclosure provides methods for high-throughput profiling one or more single nucleotides of interest in a large number of targets in situ, such as transcripts and/or DNA loci, for detecting and/or quantifying nucleic acids in cells, tissues, organs or organisms.

In some aspects, the provided methods comprise imaging the amplification product (e.g., amplicon) and/or one or more portions of the polynucleotides, for example, via binding of the detection probe and detecting the detectable label. In some embodiments, the detection probe comprises a detectable label that can be measured and quantitated. A label or detectable label can be a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. Autofluorescence can comprise background fluorescence (that can arise from a variety of sources, including aldehyde fixation, extracellular matrix components, red blood cells, lipofuscin, and the like), which is distinct from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

In some embodiments, a detectable probe containing a detectable label is used to detect one or more polynucleotide(s) and/or amplification products (e.g., amplicon) described herein. In some embodiments, the methods involve incubating the detectable probe containing the detectable label with the sample, washing unbound detectable probe, and detecting the label, e.g., by imaging.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as 125I, 35S, 14C, or 3H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. A fluorescent label can comprise a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3™-dCTP (cyanine 3-dCTP), Cy3™-dUTP (cyanine 3-dUTP), Cy5™-dCTP (cyanine 5-dCTP), Cy5™-dUTP (cyanine 5 dUTP) (Amersham Biosciences, Piscataway, N.J.), fluorescein-12-dUTP, tetramethylrhodamine-6-dUTP, TEXAS RED®-5-dUTP (red fluorescent dye-dUTP), CASCADE® BLUE-7-dUTP (blue fluorescent dye-dUTP), BODIPY™ FL-14-dUTP (green fluorescent dye-dUTP), BODIPY™ TMR-14-dUTP (orange fluorescent dye-dUTP), BODIPY™ TR-14-dUTP (red fluorescent dye-dUTP), RHODAMINE GREEN™-5-dUTP (green fluorescent dye-dUTP), OREGON GREEN™ 488-5-dUTP (green fluorescent dye-dUTP), TEXAS RED™-12-dUTP (red fluorescent dye-dUTP), BODIPY™ 630/650-14-dUTP (far red fluorescent dye-dUTP), BODIPY™ 650/665-14-dUTP (far red fluorescent dye-dUTP), ALEXA FLUOR™ 488-5-dUTP (green fluorescent dye-dUTP), ALEXA FLUOR™ 532-5-dUTP (yellow fluorescent dye-dUTP), ALEXA FLUOR™ 568-5-dUTP (red/orange fluorescent dye-dUTP), ALEXA FLUOR™ 594-5-dUTP (red fluorescent dye-dUTP), ALEXA FLUOR™ 546-14-dUTP (orange fluorescent dye-dUTP), fluorescein-12-UTP, tetramethylrhodamine-6-UTP, TEXAS RED™-5-UTP (red fluorescent dye-UTP), mCherry, CASCADE® BLUE-7-UTP (blue fluorescent dye-UTP), BODIPY™ FL-14-UTP (green fluorescent protein-UTP), BODIPY™ TMR-14-UTP (orange fluorescent dye-UTP), BODIPY™ TR-14-UTP (red fluorescent dye-UTP), RHODAMINE GREEN™-5-UTP (green fluorescent dye-UTP), ALEXA FLUOR™ 488-5-UTP (green fluorescent dye-UTP), and ALEXA FLUOR™ 546-14-UTP (orange fluorescent dye-UTP) (Molecular Probes, Inc. Eugene, Oreg.). Nucleotides having other fluorophores can also be synthesized (See, Henegariu et al. (2000) Nature Biotechnol. 18:345).

Other fluorophores comprise, but are not limited to, ALEXA FLUOR™ dyes (fluorescent dyes) such as ALEXA FLUOR™ 350 (blue fluorescent dye), ALEXA FLUOR™ 594 (red fluorescent dye), and ALEXA FLUOR™ 647 (far red fluorescent dye); BODIPY™ dyes (fluorescent dyes) such as BODIPY™ FL (green fluorescent dye), BODIPY™ TMR (orange fluorescent dye), and BODIPY™ 650/665 (far red fluorescent dye); Cascade® Blue (blue fluorescent dye), Cascade® Yellow (yellow fluorescent dye), Dansyl, lissamine rhodamine B, Marina Blue™ (blue fluorescent dye), Oregon Green™ 488, Oregon Green™ 514, Pacific Blue, rhodamine 6G, rhodamine green, rhodamine red, tetramethyl rhodamine, Texas Red® (red fluorescent dye) (available from Molecular Probes, Inc., Eugene, Oreg.), Cy2™ (cyanine 2), Cy3.5™ (cyanine 3.5), Cy5.5™ (cyanine 5.5), and Cy7™ (cyanine 7) (Amersham Biosciences, Piscataway, N.J.). FRET tandem fluorophores may also be used, comprising, but not limited to, PerCP-Cy™5.5 (far red fluorescent tandem fluorophore), PE-Cy™5 (red fluorescent tandem fluorophore), PE-Cy™5.5 (red fluorescent tandem fluorophore), PE-Cy™7 (far red fluorescent tandem fluorophore), PE-Texas Red® (red fluorescent tandem fluorophore), APC-Cy™7 (far red fluorescent tandem fluorophore), PE-Alexa™ dyes (e.g., 610, 647, 680), and APC-Alexa™ dyes

Examples of commercially available fluorescent nucleotide analogues readily incorporated into nucleotide and/or polynucleotide sequences comprise, but are not limited to, In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. In some embodiments, the term antibody comprises an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an polynucleotide sequence is indirectly labeled, especially with a hapten that is then bound by a capture agent. Example indirect labeling methods are disclosed in U.S. Pat. Nos. 5,344,757, 5,702,888, 5,354,657, 5,198,537 and 4,849,336, and 5,192,782, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any one of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity-so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequences are analyzed in situ. For example, sequences can be analyzed in situ by incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequences) of the incorporated nucleotides or labeled primer extension products can be determined, and consequently, the nucleotide sequence of the corresponding template nucleic acid. Aspects of in situ analysis are described, for example, in Mitra et al., (2003) Anal. Biochem. 320, 55-65, and Lee et al., (2014) Science, 343 (6177), 1360-1363; US 2016/0024555; US 2019/0194709; U.S. Pat. Nos. 10,138,509; 10,494,662; 10,179,932, all of which are incorporated by reference herein in their entireties.

In some cases, sequencing can be performed after the analytes are released from the biological sample. In some embodiments, sequencing is performed by sequencing-by-synthesis (SBS). In some embodiments, a sequencing primer is complementary to sequences at or near the one or more barcode(s). In such embodiments, sequencing-by-synthesis comprises reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/005986, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing is performed by sequential fluorescence hybridization. In some embodiments, sequential fluorescence hybridization is sequencing by hybridization. Sequential fluorescence hybridization can involve sequential hybridization of detection probes comprising an oligonucleotide and a detectable label.

In some embodiments, sequencing is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309:1728-1732, and in U.S. Pat. Nos. 5,599,675; 5,750,341; 6,969,488; 6,172,218; and 6,306,597, all of which are incorporated by reference herein in their entireties.

In some embodiments, the barcodes of the probes (e.g., the circularizable probe or a spatially barcoded analyte comprising a sequence of the ligated probes) or complements or products thereof are targeted by detectably labeled detection oligonucleotides, such as fluorescently labeled oligonucleotides. In some embodiments, one or more decoding schemes are used to decode the signals, such as fluorescence, for sequence determination. In some embodiments, barcodes (e.g., primary and/or secondary barcode sequences) can be analyzed (e.g., detected or sequenced) using any suitable methods or techniques. In some embodiments, the barcodes are primary and/or secondary barcode sequences. In some embodiments, analysis comprises detection or sequencing.

In some embodiments, nucleic acid hybridization is used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are incorporated by reference herein in their entireties.

In some embodiments, real-time monitoring of DNA polymerase activity is used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181, all of which are incorporated by reference herein in their entireties.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### V. Sample Preparation

A sample disclosed herein can be or be derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaca, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, a cell pellet, a cell block, a needle aspirate, or fine needle aspirate. The sample can be a fluid sample, such as a blood sample, urine sample, or saliva sample. The sample can be a skin sample, a colon sample, a check swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, a substrate herein is any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In some embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some embodiments, the biological sample (e.g., a tissue section as described above) is prepared by deep freezing at a temperature suitable to maintain or preserve the integrity (e.g., the physical characteristics) of the tissue structure. In some embodiments, the biological sample is a tissue section as described above. In some embodiments, the integrity of the tissue structure includes physical characteristics. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample is prepared using formalin-fixation and paraffin-embedding (FFPE), which are established methods. In some embodiments, cell suspensions and other non-tissue samples are prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, the methods provided herein comprises one or more post-fixing (also referred to as postfixation) steps. In some embodiments, one or more post-fixing steps are performed after contacting a sample with a polynucleotide disclosed herein, e.g., one or more probes such as a circular or circularizable probe. In some embodiments, one or more post-fixing step is performed after a hybridization complex comprising a probe and a target is formed in a sample. In some embodiments, one or more post-fixing step is performed prior to a ligation reaction disclosed herein.

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

In some embodiments, a biological sample is permeabilized to facilitate transfer of species (such as probes) into the sample. In some embodiments, the species are probes. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample is permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, are added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ii) Embedding

In some embodiments, the biological sample is embedded in a matrix. In some embodiments, the matrix is a hydrogel matrix. Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, the amplicons are rolling circle amplification products. In some embodiments, the analytes are protein, RNA, and/or DNA. In some embodiments, a 3D matrix comprises a network of natural molecules and/or synthetic molecules that are chemically and/or enzymatically linked. In some embodiments, the chemical and/or enzymatic linking is crosslinking. In some embodiments, a 3D matrix comprises a synthetic polymer. In some embodiments, a 3D matrix comprises a hydrogel.

In some aspects, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof is modified to contain functional groups that are used as an anchoring site to attach the polynucleotide probes and/or amplification product to a polymer matrix. In some embodiments, a modified probe comprising oligo dT is used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel includes hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly (hydroxyethyl acrylate), and poly (hydroxyethyl methacrylate), collagen, hyaluronic acid, chitosan, dextran, agarose, gelatin, alginate, protein polymers, methylcellulose, and the like, and combinations thereof. In some embodiments, the poly(ethylene glycol) and derivatives thereof are PEG-acrylate (PEG-DA) and/or PEG-RGD.

In some embodiments, a hydrogel includes a hybrid material. For example, the hydrogel material can include elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347 (6221): 543-548, 2015, the entire contents of which are incorporated herein by reference.

In some embodiments, the hydrogel forms the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, the biological sample is a tissue section. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible. In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some embodiments, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums). In some embodiments, the medium is a mounting medium, methylcellulose, or other semi-solid mediums.

In some embodiments, a biological sample embedded in a matrix (e.g., a hydrogel) is isometrically expanded. In some embodiments, the matrix is a hydrogel matrix. Isometric expansion methods that can be used include hydration, a preparative step in expansion microscopy, as described in, e.g., Chen et al., Science 347 (6221): 543-548, 2015 and U.S. Pat. No. 10,059,990, all of which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(iii) Staining and Immunohistochemistry (IHC)

To facilitate visualization, biological samples can be stained using a wide variety of stains and staining techniques. For example, a sample can be stained using any number of stains and/or immunohistochemical reagents. One or more staining steps may be performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample are segmented using one or more images taken of the stained sample.

In some embodiments, the stain is performed using a lipophilic dye. In some examples, the staining is performed with a lipophilic carbocyanine or aminostyryl dye, or analogs thereof (e.g., DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, cosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample is stained with haematoxylin and cosin (H&E).

The sample can be stained using hematoxylin and cosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample is stained using Romanowsky stain, including Wright's stain, Jenner's stain, Can-Grunwald stain, Leishman stain, and Giemsa stain.

In some embodiments, biological samples are destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65 (8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### VI. Compositions, Kits, and Systems

In some embodiments, disclosed herein is a composition that comprises a composition containing a target nucleic acid and one or more probes or probe sets comprising a non-phosphorylated 5′ end. In some embodiments, disclosed herein is a composition that comprises an amplification product containing monomeric units of a sequence complementary to a sequence of a probe (e.g., a circularizable probe). In some embodiments, the amplification product is formed using any one of the target nucleic acids, probes (e.g., circularizable probes) and any one of the amplification techniques described herein. In some embodiments, the probe is a circularizable probe.

Also provided herein are kits, for example comprising one or more polynucleotides (circularizable probes or probe sets each comprising at least a first and second probe), e.g., any described in Section II, and reagents for performing the methods provided herein, for example reagents required for one or more steps comprising hybridization, phosphorylation, ligation, amplification, detection, sequencing, and/or sample preparation as described herein. In some embodiments, the kit further comprises a target nucleic acid. In some embodiments, any or all of the polynucleotides are DNA molecules. In some embodiments, the target nucleic acid is a messenger RNA molecule. In some embodiments, the kit further comprises a kinase (e.g., a PNK). In some embodiments, the kinase is a PNK. In some embodiments, the kit further comprises a ligase, for instance for forming a ligated circularized probe from the circularizable probe (e.g., padlock probe) or probe set. In some embodiments, the ligase has DNA-splinted DNA ligase activity. In some embodiments, the kit further comprises a polymerase, for instance for performing amplification of the circularizable probe. In some embodiments, the polymerase is capable of using the ligated circular probe as a template for amplification. In some embodiments, the kit further comprises a primer for amplification.

In some embodiments, the kits further comprise one or more reagents for array based analysis, such as capture probes, e.g., any described in Section III. For example, provided are kits comprising a substrate (e.g., an array) with a plurality of oligonucleotides immobilized thereon. For example, the oligonucleotides immobilized on the substrate each comprise a spatial barcode sequence and a capture sequence complementary to a sequence of the ligated probe and/or the product thereof (e.g., described in Section II).

Disclosed herein in some aspects is a kit comprising a probe or probe set, and the molecules of the probe or probe set lack phosphorylated 5′ ends and a kinase for phosphorylating one or more ends of the probe or probe set hybridized to a target nucleic acid.

In some embodiments, the kit further comprises reagents for amplifying the circular probe using rolling circle amplification (RCA) to generate an RCA product; and for detecting the RCA product in the biological sample.

In some aspects, disclosed herein is a kit comprising a plurality of circularizable probes for use in a method for analyzing a biological sample comprising a plurality of target RNA molecules, the method comprising: a) contacting the biological sample with a plurality of circularizable probes, each comprising a hybridization region that hybridizes to the target region in the cell or tissue sample, and wherein upon hybridization of the circularizable probe to the target nucleic acid, the sample is treated with a kinase to phosphorylate the 5′ end(s) of the hybridized circularizable probes, ligating the circularizable probe to generate a circularized probe using the target nucleic acid as template; and amplifying the circularized probe using rolling circle amplification (RCA) to generate an RCA product; and detecting the RCA product in the biological sample. In some embodiments, if the circularizable probe is provided in two or more parts, the kit comprises an additional splint nucleic acid to template one or more additional ligations for generating the circularized probe.

The various components of the kit may be present in separate containers or certain compatible components may be pre-combined into a single container. In some embodiments, the kits further contain instructions for using the components of the kit to practice the provided methods.

In some embodiments, the kits contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any one of the reagents described herein, e.g., wash buffer and ligation buffer. In some embodiments, the kits contain reagents for detection and/or sequencing, such as barcode detection probes or detectable labels. In some embodiments, the kits contain other components, for example nucleic acid primers, enzymes and reagents, buffers, nucleotides, modified nucleotides, reagents for additional assays.

### VII. Applications

In some aspects, the provided embodiments can be applied in an in situ method of analyzing nucleic acid sequences, such as an in situ transcriptomic analysis, for example from intact tissues or samples in which the spatial information has been preserved. In some aspects, the embodiments can be applied in a spatial array. In some aspects, the embodiments can be applied in a single-cell profiling assay. In some aspects, the embodiments can be applied in an imaging or detection method for multiplexed nucleic acid analysis. In some aspects, the provided embodiments can be used to identify or detect regions of interest in target nucleic acids.

In some embodiments, the region of interest comprises more than one nucleotide of interest. In some embodiments, the region of interest is a single nucleotide of interest. In some embodiments, the single nucleotide of interest is a single-nucleotide polymorphism (SNP). In some embodiments, the single nucleotide of interest is a single-nucleotide variant (SNV). In some embodiments, the single nucleotide of interest is a single-nucleotide substitution. In some embodiments, the single nucleotide of interest is a point mutation. In some embodiments, the single nucleotide of interest is a single-nucleotide insertion. In some embodiments, the single nucleotide of interest is a single-nucleotide deletion.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VIII. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule,” used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

In some aspects, the term “about” as used herein refers to the usual error range for the respective value readily known to the skilled person in this technical field. Reference to “about” a value or parameter herein comprises (and describes) embodiments that are directed to that value or parameter per se. In some instances, the term “about” can refer to a value within 20% of an indicated value.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: In Situ Phosphorylation of Hybridized Probes Facilitates Probe Ligation and Detection of Target Nucleic Acids

This example demonstrates a workflow involving in situ phosphorylation and ligation of a probe or probe set contacted with a biological sample to detect a target nucleic acid.

Circularizable probes were contacted with a fresh frozen mouse brain tissue sample, and incubated in hybridization buffer to allow for probe hybridization. The circularizable probes tested were linear nucleic acid molecules each comprising a split hybridization region for binding to the target nucleic acid wherein a first hybridization region at the 5′ end of the circularizable probe molecule binds a first sequence in the target nucleic acid and a second hybridization region at the 3′ end of the circularizable probe molecule binds a second sequence in the target nucleic acid that is adjacent to the first sequence in the target nucleic acid. After hybridization, the samples were washed and incubated at 37° C. for 1 hour to perform in situ phosphorylation of the probe and probe sets using T4 PNK. For comparison, probes synthesized with a 5′ phosphate modification (“5′ Phos Mod”) were also tested. Probes synthesized with a 5′ phosphate modification and then treated with CIP (calf intestinal alkaline phosphatase) to remove phosphates prior to ligation and rolling circle amplification, were also tested. After phosphorylation, the sample was washed and a ligation was performed with a ligase using the target nucleic acid as template. The sample was incubated with a rolling-circle amplification (RCA) mixture containing a Phi29 DNA polymerase and dNTP at approximately 30° C. for RCA of the circularized probes. For detection, fluorescently labeled oligonucleotides were used to hybridize to overhang regions of intermediate probes, and each intermediate probe comprised a hybridization region complementary to a portion of an amplification product of a circularized probe (e.g., barcodes of the RCA product associated with the target nucleic acid), and after washing, images were obtained to detect the fluorescently labeled oligonucleotides. Multiple cycles of hybridization were performed and images were acquired each cycle using a confocal microscope.

In one experiment, single molecule circularizable probes synthesized with a 5′ phosphate modification were hybridized to a target nucleic acid in a biological sample. In a first experimental condition (FIG. 2A; left), the circularizable probes were treated with a ligase, amplified by RCA, and detected via hybridization and fluorescence microscopy, generally as described above. A clear signal corresponding to the hybridized probes was detected. In a second condition (FIG. 2A; middle), the probes were treated with CIP to remove the 5′ phosphate, and subsequent steps for ligation, amplification, and detection were performed. The signal corresponding to the hybridized probes was abolished in the condition with the probes lacking a 5′ phosphate. In a third condition (FIG. 2A; right), following CIP treatment to remove the 5′ phosphate, hybridized probes were treated with T4 PNK in situ to introduce a 5′ phosphate required for ligation, and subsequent steps for ligation, amplification, and detection were performed. In situ phosphorylation of the probes in the third condition facilitated detection of a clear signal corresponding to the hybridized probes. A similar experiment was performed using a circularizable probe set comprising a first and second probe that each contained a region for hybridizing to adjacent target sequences in the target nucleic acid. For the circularizable probe set, a first ligation was templated by the RNA target nucleic acid, and a second ligation was templated by a splint nucleic acid. Similar to the results for circularizable probes, in situ phosphorylation of previously CIP-treated circularizable probe sets facilitated detection of a signal corresponding to the hybridized probes. The results indicate that probes or probe sets lacking 5′ phosphates can be hybridized and phosphorylated in situ in accordance with the methods described herein to facilitate downstream ligation and detection.

As shown in FIG. 2B, different wash conditions (PBST or ligation buffer) were tested prior to the PNK phosphorylation step with either 1 circularizable probe molecule (top row) or 2 probe molecules that form the circularizable probe set (bottom row). Washes performed with the ligation buffer (e.g., comprising water, Tris pH 8.0, MgCl₂, KCl, ATP, DTT, glycerol, and BSA) prior to PNK treatment generated detected signals consistent with higher sensitivity in the assay.

### Example 2: In Situ Phosphorylation of Hybridized Probes Facilitates Probe Ligation and Detection of Target Nucleic Acids in Multiplexed Detection Assays

This example demonstrates workflows involving in situ phosphorylation and ligation of probes or probe sets contacted with a biological sample to detect a plurality of target nucleic acids in multiplexed assays.

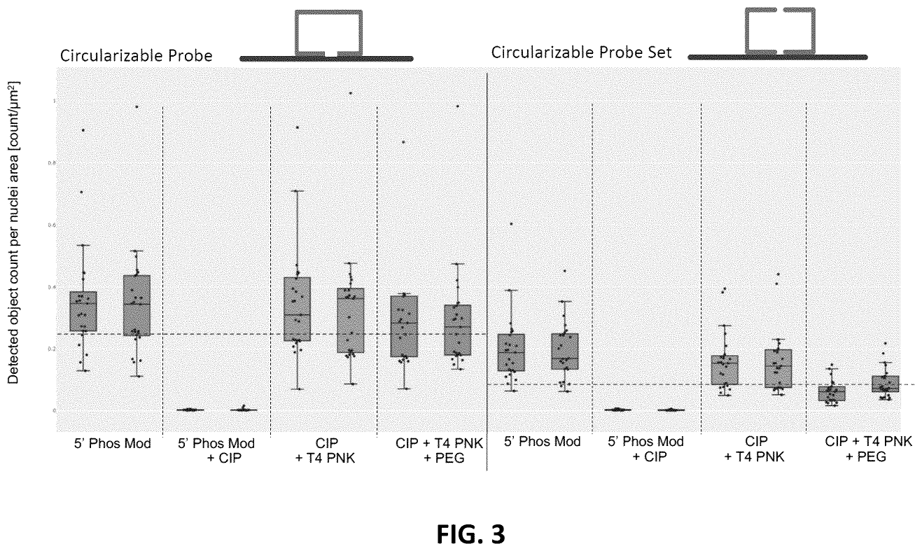

A sample was contacted with a mixture of circularizable probes (FIG. 3; left) or a mixture of circularizable probe sets each with two probe molecules (FIG. 3; right), with each mixture designed to target a panel of 24 different target nucleic acids (e.g., mRNAs) to further test in situ phosphorylation of a plurality of probes in a sample for detection of a plurality of target nucleic acids. FIG. 3 shows results of detected amplification products observed with the indicated circularizable probes or circularizable probe sets and treatments. The results show that while removal of 5′ phosphate by CIP abolished ligation and signal detection, subsequent in situ phosphorylation of hybridized probes facilitated ligation and signal detection at a level similar to positive control conditions using 5′ phosphorylated probes without CIP treatment. Thus, in situ phosphorylation was observed to be useful in generating circularized probes that were amplified and detected in the tissue sample, in an assay for detection of a plurality of target nucleic acids in situ.

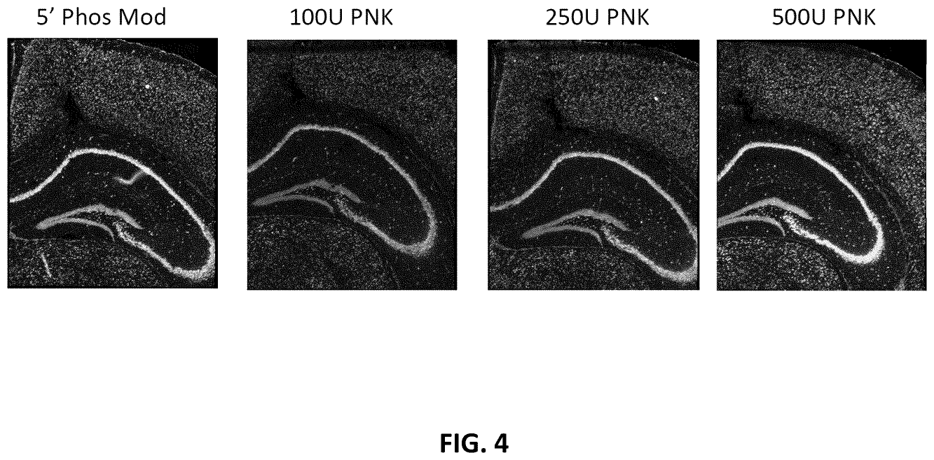

In another experiment, in situ phosphorylation of circularizable probes targeting different target nucleic acid analytes in a larger panel of approximately 250 genes was performed, followed by ligation, amplification, and detection steps. Analysis of signal detection across the entire panel indicated that in situ phosphorylation of the circularizable probes consistently resulted in comparable detection levels to positive control conditions for almost all analytes. FIG. 4 shows exemplary results from 5′ phosphorylated probes from the panel (positive control; left), and results from CIP-treated probes lacking 5′ phosphates that were subsequently hybridized and phosphorylated in situ using the indicated concentrations of T4 PNK. The figure shows exemplary signals detected in situ. The results demonstrate effective phosphorylation of circularizable probes in situ across a range of T4 PNK concentrations.

Taken together, the results show that probes or probe sets lacking 5′ phosphates can be hybridized to targets and phosphorylated in situ in accordance with the methods described herein to facilitate downstream ligation and detection in highly multiplexed assays for detecting different target analytes in a biological sample. The results also show that in situ phosphorylation of nucleic acid probes in an assay involving downstream ligation and detection can be used to significantly reduce assay costs while not significantly impacting assay performance and detection (e.g., in comparison to generating nucleic acid probes with phosphate modifications in vitro).

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method, comprising:
a) contacting a target nucleic acid in a cell or tissue sample with a probe or probe set comprising a non-phosphorylated 5′ end, wherein:
the target nucleic acid comprises a target region and the probe or probe set comprises a hybridization region, and the hybridization region hybridizes to the target region in the cell or tissue sample;
b) contacting the cell or tissue sample with a kinase to phosphorylate the non-phosphorylated 5′ end of the probe or probe set hybridized to the target nucleic acid;
c) ligating the probe or probe set to form a circularized probe in the cell or tissue sample; and
d) detecting the circularized probe or an amplification product thereof.

2. The method of claim 1, wherein ligation of the probe or probe set comprises a RNA templated ligation and/or a DNA templated ligation.

3. (canceled)

4. The method of claim 1, further comprising performing a wash to remove unbound probes or probe sets prior to contacting the cell or tissue sample with the kinase.

5-7. (canceled)

8. The method of claim 1, wherein the phosphorylation of the non-phosphorylated 5′ end of the probe or probe set is performed in a buffer comprising ATP.

9-10. (canceled)

11. The method of claim 1, wherein the cell or tissue sample is incubated with the kinase for at least 30 minutes.

12. The method of claim 1, wherein the kinase is a T7 or T4 Polynucleotide Kinase (PNK).

13. The method of claim 1, wherein the probe is a linear probe comprising a split hybridization region comprising a first hybridization region at the 5′ end of the probe and a second hybridization region at the 3′ end of the probe, wherein the ligating the probe comprises ligating the 5′ end to the 3′ end.

14. The method of claim 1, wherein the probe set comprises a first probe and a second probe, wherein the hybridization region of the probe set comprises a split hybridization region comprising a first hybridization region comprised by the first probe of the probe set and a second hybridization region comprised by the second probe of the probe set.

15. (canceled)

16. The method of any of claim 1, further comprising contacting the cell or tissue sample with a splint nucleic acid for ligating the probe or probe set.

17-21. (canceled)

22. The method of claim 16, wherein ligation of the probe set comprises two or more ligations, and wherein the two or more ligations comprise: a first ligation wherein a 5′ end of the first probe is ligated to a 3′ end of the second probe using the target nucleic acid as a first template, and a second ligation wherein a 3′ end of the first probe is ligated to a 5′ end of the second probe using the splint nucleic acid as a second template.

23. (canceled)

24. The method of claim 1, wherein the probe or probe set comprises one or more barcode sequences, wherein the amplification product comprises the one or more barcode sequences or complementary sequences thereof, and wherein the detecting the amplification product of the circularized probe comprises detecting the one or more barcode sequences or complementary sequences thereof.

25-27. (canceled)

28. The method of claim 1, wherein the cell or tissue sample comprises a plurality of target nucleic acids and the cell or tissue sample is contacted with a plurality of probes or probe sets, wherein each probe or probe set of the plurality of probes or probe sets corresponds to a target nucleic acid of the plurality of target nucleic acids, and wherein the method comprises detecting circularized probes or amplification products thereof generated from the plurality of probes or probe sets.

29-33. (canceled)

34. The method of claim 1, wherein the target nucleic acid is at a location in the cell or tissue sample and the amplification product of the circularized probe is generated and detected at the location in the cell or tissue sample.

35. A method, comprising:
a) contacting a cell or tissue sample with a plurality of probe sets, wherein each probe set of the plurality of probe sets comprises a first probe and a second probe, wherein the target nucleic acid comprises a first target region and a second target region and the first and second probes hybridize to the first and second target regions, respectively;
b) contacting the cell or tissue sample with a kinase to phosphorylate the 5′ ends of the first probes and/or the 5′ ends of the second probes hybridized to the target nucleic acid;
c) ligating the first probes to the second probes hybridized to the target nucleic acid to form a plurality of ligated probes in the cell or tissue sample; and
d) detecting the plurality of ligated probes or a product thereof.

36-54. (canceled)

55. The method of claim 35, wherein the plurality of ligated probes or a product thereof is detected on an array and/or by nucleic acid sequencing.

56. The method of claim 35, wherein the target nucleic acid is at a location in the cell or tissue sample and a ligated probe of the plurality of ligated probes is generated at the location in the cell or tissue sample, and wherein the ligated probe of the plurality of ligated probes and/or a product thereof is detected at the location in the cell or tissue sample.

57. The method of claim 35, wherein the target nucleic acid is at a location in the cell or tissue sample and a ligated probe of the plurality of ligated probes is generated at the location in the cell or tissue sample, and wherein the ligated probe of the plurality of ligated probes and/or the product thereof is covalently or noncovalently attached to an oligonucleotide immobilized on a substrate, wherein the oligonucleotide comprises a spatial barcode sequence.

58-62. (canceled)

63. The method of claim 1, wherein the target nucleic acid is a cellular target nucleic acid or a product thereof.

64-68. (canceled)

69. The method of claim 1, wherein the target nucleic acid is comprised in a labelling agent that directly or indirectly binds to an analyte in the sample or is comprised in a product of the labelling agent.

70-76. (canceled)

77. The method of claim 1, wherein the detecting comprises imaging the cell or tissue sample to detect signals associated with the target nucleic acid in situ.

78-79. (canceled)
